(19) United States
(12) Patent Application Publication
(10) Pub. No.: US 2026/0012764 A1
(43) Pub. Date: Jan. 8, 2026

(54) ENVIRONMENTAL DATA HUB

(71) Applicant: Aclima Inc., San Leandro, CA (US)

(72) Inventors: Salvatore Mazzola, San Francisco, CA (US); Aja Ellis, Seattle, WA (US); Garrett Brown, Ventura, CA (US); Amol Shirke, Fremont, CA (US); Jeff Pao, Palo Alto, CA (US)

(21) Appl. No.: 19/183,403

(22) Filed: Apr. 18, 2025

Related U.S. Application Data
(60) Provisional application No. 63/637,305, filed on Apr. 22, 2024.

(51) Int. Cl. H04W 4/38; G01N 33/00; H04W 84/18

(52) U.S. Cl. CPC H04W 4/38; G01N 33/0075; H04W 84/18

(57) ABSTRACT

A system, device, and method for collecting and processing sensor data at an edge system is disclosed. The system includes (i) a bridge configured to receive time series sensor data sets from a plurality of environmental sensors and having a plurality of formats, and (ii) a data hub, coupled with the bridge, configured to parse each of the time series data sets, determine a sensor type and data format for each of the plurality of environmental sensors, and convert the data format to a predefined format.

### CROSS REFERENCE TO OTHER APPLICATIONS

This application claims priority to U.S. Provisional Patent Application No. 63/637,305 entitled ENVIRONMENTAL DATA HUB filed Apr. 22, 2024 which is incorporated herein by reference for all purposes.

### BACKGROUND OF THE INVENTION

Monitoring of environmental conditions includes measuring the levels of various components of the surroundings, allowing detection of potentially harmful air pollution, radiation, greenhouse gases, or other contaminants in the environment. Depending on the application, environmental monitoring systems can be used in outdoor or indoor settings. Monitoring of environmental conditions typically includes gathering environmental data. Environmental data includes detection and measurement of pollutants or contaminants such as nitrogen dioxide (NO₂), carbon monoxide (CO), nitrogen oxide (NO), ozone (O₃), sulfur dioxide (SO₂), carbon dioxide (CO₂), methane (CH₄), volatile organic compounds (VOC), air toxics, temperature, sound radiation, and particulate matter. In order to assess the effects of such pollutants, it is desirable to associate environmental data sensing these pollutants at particular times with geographic locations (homes, businesses, towns, etc.). Such an association would allow individuals and communities to evaluate the quality of their surroundings. Thus, data collected that is representative of the region is desired to be collected. Further, the data collected is desired to meet desired error tolerances, and be collected and processed efficiently. Thus, a mechanism for improving collection and processing of environmental data is desired.

### BRIEF DESCRIPTION OF THE DRAWINGS

Various embodiments of the invention are disclosed in the following detailed description and the accompanying drawings.

FIG. 1 illustrates an embodiment of a system for capturing environmental data using mobile sensor platforms and associating the environmental data with map features.

FIG. 2 illustrates an embodiment of a method for capturing environmental data using mobile sensor platforms.

FIGS. 3A-3C illustrate a particular region and the embodiment of routes that may be traversed using a method for capturing environmental data using mobile sensor platforms.

FIG. 4 illustrates an embodiment of an edge system for collecting and analyzing environmental data.

FIGS. 5A and 5B illustrate examples of data collection paths.

FIGS. 6A and 6B illustrate examples of data collection paths.

FIGS. 7A-7C illustrate examples of user interfaces for displaying visualizations of environmental data based on real-time analysis of sensor data collected at a data hub according to various embodiments.

FIG. 8 illustrates an example of a visualization of data collected via a broad monitoring of a predefined region according to various embodiments.

FIG. 9 illustrates a method for collecting environmental data at an edge system according to various embodiments.

FIG. 10 illustrates a method for collecting and storing environmental data at an edge system according to various embodiments.

FIG. 11 illustrates a method for performing an environmental data analysis locally at an edge system according to various embodiments.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

In many conventional environmental monitoring setups, organizations rely on an assortment of sensors for collecting data related to temperature, air quality, and other environmental parameters. These legacy or traditional systems are often rigid, requiring engineers to manually integrate each new sensor type through customized code or configuration files. The process can be time-consuming and expensive, as developers must carefully study the sensor's communication protocol, data format, and power or voltage requirements to ensure that data is properly collected. Each additional sensor type typically demands unique adjustments to the system, resulting in software overhead, extended development timelines, and greater cost for organizations. When dealing with large sensor networks or frequent updates to monitoring hardware, such a setup can become unwieldy and prone to errors.

In many environmental monitoring and data collection initiatives, organizations and agencies rely on specialized sensors to gather information about various parameters. These sensors can measure temperature, precipitation, hydrogen sulfide concentrations, water quality, soil composition, and other characteristics relevant to understanding pollution, air quality, and broader ecological impacts. The environmental data may be collected using static sensor systems and/or mobile environmental platforms, such as environmental platforms mounted to a vehicle. Hyper-local environmental data, for example, related to air quality and greenhouse gas data, can be collected using vehicles with air pollutant sensors installed. Embodiments of techniques usable in gathering hyper-local data are described in U.S. patent application Ser. No. 16/682,871, filed on Nov. 13, 2019, entitled HYPER-LOCAL MAPPING OF ENVIRONMENTAL CONDITIONS and assigned to the assignee of the present application, U.S. patent application Ser. No. 16/409,624, filed on May 10, 2019, entitled INTEGRATION AND ACTIVE FLOW CONTROL FOR ENVIRONMENTAL SENSORS and assigned to the assignee of the present application; U.S. patent application Ser. No. 16/773,873, filed on Jan. 27, 2020, entitled SENSOR AND DATA PLATFORMS FOR VEHICLE ENVIRONMENTAL QUALITY MANAGEMENT, assigned to the assignee of the present application and which claims priority to U.S. Patent Application Ser. No. 62/798,395 entitled SENSOR AND DATA PLATFORM FOR VEHICLE ENVIRONMENTAL QUALITY MANAGEMENT and assigned to the assignee of the present application, which are all incorporated herein in their entirety for all purposes.

Conventional systems also tend to rely heavily on centralized or remote data processing. In many cases, the sensor data is uploaded to the cloud or routed to a server rack maintained by the organization, where the measurements are stored in databases and processed offsite. Although this approach can handle significant volumes of data, it introduces latency, consumes high bandwidth, and may result in slow turnaround times for analysis or real-time monitoring. Additionally, organizations lacking robust network connectivity or cloud infrastructure can find themselves unable to perform on-demand analytics, leaving them with large amounts of data that cannot be readily visualized or utilized for immediate decision-making. As a result, traditional systems often do not support real-time, localized data processing and thus cannot offer immediate feedback or insights in resource-constrained environments. Furthermore, many agencies and organizations do not possess cloud-based infrastructure, nor do they have the resources or flexibility to deploy or update complex platforms for collecting and analyzing sensor data in real time. As a result, there is a need for an efficient, edge-based solution that can handle the volume and velocity of continuous sensor data streams in an adaptable and cost-effective manner.

Various embodiments provide a system, device, and method for collecting and analyzing sensor data, such as at an edge system (e.g., locally at the environmental platform). The system includes (i) a bridge configured to receive time series sensor data sets from a plurality of environmental sensors and having a plurality of formats, and (ii) a data hub, coupled with the bridge, configured to parse each of the time series data sets, determine a sensor type and data format for each of the plurality of environmental sensors, and convert the corresponding sensor data (e.g., the sensor data collected by a particular sensor of the plurality of environmental sensors) to a predefined format.

In some embodiments, the method includes: (a) receiving, at an edge system, sensor data from a particular environmental sensor of a plurality of environmental sensors connected to the edge system, (b) determining an environmental data type based at least in part on one or more of the sensor data and sensor information detected based on a connection between the particular environmental sensor and the edge system, (c) parsing the sensor data based at least in part on the environmental data type, and (d) causing environmental analysis data to be displayed.

According to various embodiments, the system operates as a middleware or bridge that is deployed locally to ingest and process data from various sensors without relying heavily on cloud-based infrastructures. By performing real-time data processing at the edge, the system can provide immediate insights and data visualization. This real-time capability is particularly beneficial in regulatory or agency contexts where constraints on network access or funding make cloud computing either unavailable or prohibitively expensive. As a local hub, the system can autonomously handle tasks such as parsing incoming time series data, detecting the connected sensors and their communication protocols, and providing a uniform time stamp and geolocation reference that applies across diverse sensor inputs. By integrating a local clock and geolocation service (for example, GPS), the system ensures consistency in the recorded measurements while simplifying the downstream handling of the data.

In some embodiments, the system is configured to automatically detect sensor type or model. The system can automatically detect the sensor type or model based at least in part on one or more of referencing data profiles, detecting the nature of the cables or ports used, and/or recognizing identifiers embedded in the data streams. This automated detection can eliminate the need to write and maintain custom code for each new sensor model or data protocol. Once the system identifies a new sensor, it can interpret the incoming measurements using a stored set of configuration files that specify data formats, communication parameters, voltage levels, and other relevant variables. This allows the system to handle a variety of sensors—including those for air quality monitoring and water sampling-without requiring extensive manual setup or specialized programming knowledge. The real-time processing component then enables the immediate/real-time identification of certain events, such as peaks in pollutant concentrations, which can be critical in environmental monitoring and rapid response scenarios.

The system supports local data storage and provides convenient channels for uploading data either to a connected USB device or eventually to the cloud if such resources become available. Because the system in some implementations can be deployed as a middleware layer between multiple sensors and an environmental platform or service, the system can aggregate sensor data and perform analytics that are typically associated with centralized infrastructure. In doing so, the system can save on bandwidth usage and cloud compute resources. By ingesting, analyzing, and visualizing the data locally, the system empowers users to spot anomalies, sensor errors, or significant changes in measurement values as they arise, rather than waiting for data post-processing that might otherwise occur hours or days later in a remote server.

In some embodiments, the system can perform real-time analysis and visualization of sensor data analysis results, such as environmental monitoring data. For visualization purposes, the system can format its outputs for display on standard devices such as tablets or laptops, making it particularly versatile for fieldwork and immediate data interpretation. Users can leverage familiar graphics tools, including those found in software libraries and frameworks like matplotlib, Plotly, or other Python-based libraries, to plot sensor readings and highlight trends. The system may implement built-in peak detection algorithms to enable identification of sudden surges of pollutants or other environmental measurements (e.g., surges in gas concentrations, such as peaks in carbon dioxide) and the system can correlate these peaks with other sensor streams, and flag potential issues in real-time for immediate action. In some embodiments, the system comprises a GPS module that obtains GPS data associated with the sensor locations. If geolocation data is available, the system can map these readings to geographical coordinates and provide near real-time spatial analytics, allowing quicker decisions and more informed resource allocations in environmental monitoring applications.

Through these integrated capabilities, the system substantially improves upon existing sensor-based data collection methodologies by reducing reliance on external cloud services, speeding up analysis, and adapting easily to new or evolving sensor technologies.

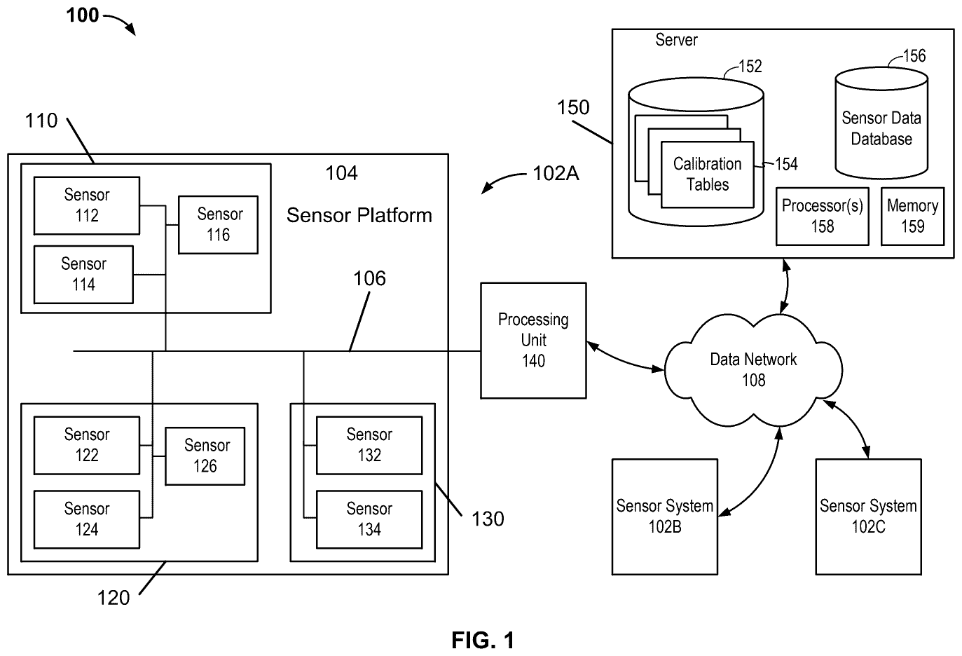

FIG. 1 depicts an embodiment of a system 100 for collecting and processing environmental data. System 100 includes multiple mobile sensor platforms 102A, 102B, 102C and server 150. In some embodiments, system 100 may also include one or more stationary sensor platforms 103, of which one is shown. Stationary sensor platform 103 may be used to collect environmental data at a fixed location. The environmental data collected by stationary sensor platform 103 may supplement the data collected by mobile sensor platforms 102A, 102B and 102C. Thus, stationary sensor platform 103 may have sensors that are the same as or analogous to the sensors for mobile sensor platforms 102A, 102B and 102C. In other embodiments, stationary sensor platform 103 may be omitted. Although a single server 150 is shown, multiple servers may be used. The multiple servers may be in different locations. Although three mobile sensor platforms 102A, 102B and 102C are shown, another number are typically present. Mobile sensor platforms 102A, 102B and 102C and stationary sensor platform(s) 103 may communicate with server 150 via a data network 108. The communication may take place wirelessly.

Mobile sensor platforms 102A, 102B and 102C may be mounted in a vehicle, such as an automobile or a drone. In some embodiments, mobile sensor platforms 102A, 102B and 102C are desired to stay in proximity to the ground to be better able to sense conditions analogous to what a human would experience. Mobile sensor platform 102A includes a bus 106, sensors 110, 120 and 130. Although three sensors are shown, another number may be present on mobile sensor platform 102A. In addition, a different configuration of components may be used with sensors 110, 120 and 130. Each sensor 110, 120 and 130 is used to sense environmental quality and may be of primary interest to a user of system 100. For example, sensors 110, 120 and 130 may be gas sensors, volatile organic compound (VOC) sensors, particulate matter sensors, radiation sensors, noise sensors, light sensors, temperature sensors, noise sensors or other analogous sensors that capture variations in the environment. For example, sensors 110, 120 and 130 may be used to sense one or more of NO2, CO, NO, O3, SO2, CO2, VOCs, CH4, particulate matter, noise, light, temperature, radiation, and other compounds. In some embodiments, sensor 110, 120 and/or 130 may be a multi-modality sensor. A multi-modality gas sensor senses multiple gases or compounds. For example, if sensor 110 is a multi-modality NO2/O3 sensor, sensor 110 might sense both NO2 and O3 together. Sensor 110 may comprise a plurality of sensors, such as sensors 112, 114, and 116. Sensor 120 may comprise a plurality of sensors, such as sensors 122, 124, and 126. Sensor 130 may comprise a plurality of sensors, such as sensors 132 and 134.

Although not shown in FIG. 1, other sensors co-located with sensors 110, 120 and 130 may be used to sense characteristics of the surrounding environment including, in some instances, other gases and/or matter. Such additional sensors are exposed to the same environment as sensors 110, 120 and 130. In some embodiments, such additional sensors are in close proximity to sensors 110, 120 and 130, for example within ten millimeters or less. In some embodiments, the additional sensors may be further from sensors 110, 120 and 130 if the additional sensors sample the same packet of air inside of a closed system, such as a system of closed tubes. In some embodiments, temperature and/or pressure are sensed by these additional sensors. For example, an additional sensor co-located with sensor 110 may be a temperature, pressure, and relative humidity (T/P/RH) sensor. These additional co-located sensors may be used to calibrate sensors 110, 120 and/or 130. Although not shown, sensor platform 102A may also include a manifold for drawing in air and transporting air to sensors 110, 120 and 130 for testing.

Sensors 110, 120 and 130 provide sensor data over bus 106, or via another mechanism. In some embodiments, data from sensors 110, 120 and 130 incorporates time. This time may be provided by a master clock (not shown) and may take the form of a timestamp. Master clock may reside on sensor platform 102A, may be part of processing unit 140, or may be provided from server 150. As a result, sensors 110, 120 and 130 may provide timestamped sensor data to server 150. In other embodiments, the time associated with the sensor data may be provided in another manner. Because sensors 110, 120 and 130 generally capture data at a particular frequency, sensor data is discussed as being associated with a particular time interval (e.g., the period associated with the frequency), though the sensor data may be timestamped with a particular value. For example, sensors 110, 120 and/or 130 may capture sensor data every second, every two seconds, every ten seconds, or every thirty seconds. The time interval may be one second, two seconds, ten seconds, or thirty seconds. The time interval may be the same for all sensors 110, 120 and 130 or may differ for different sensors 110, 120 and 130. In some embodiments, the time interval for a sensor data point is centered on the timestamp. For example, if the time interval is one second and a timestamp is t1, then the time interval may be from t1−0.5 seconds to t1+0.5 seconds. However, other mechanisms for defining the time interval may be used.

Sensor platform 102A also includes a position unit 145 that provides position data. In some embodiments, position unit 145 is a global positioning satellite (GPS) unit. Consequently, system 100 is described in the context of a position unit 145. The position data may be time-stamped in a manner analogous to sensor data. Because position data is to be associated with sensor data, the position data may also be considered associated with time intervals, as described above. However, in some embodiments, position data (e.g., GPS data) may be captured more or less frequently than sensor data. For example, position unit 145 may capture position data every second, while sensor 130 may capture data every thirty seconds. Thus, multiple data points for the position data may be associated with a single thirty second time interval. The position data may be processed as described below.

Optional processing unit 140 may perform some processing and functions for data from sensor platform 104, may simply pass data from sensor platform 104 to server 150 or may be omitted.

Mobile sensors platforms 102B and 102C are analogous to mobile sensor platform 102A. In some embodiments, mobile sensor platforms 102B and 102C have the same components as mobile sensor platform 102A. However, in other embodiments, the components may differ. However, mobile sensor platforms 102A, 102B and 102C function in an analogous manner.

Server 150 includes sensor data database 156, calibration tables 154 (e.g., stored in database 152), processor(s) 158, memory 159. Processor(s) 158 may include multiple cores. Processor(s) 158 may include one or more central processing units (CPUs), one or more graphical processing units (GPUs) and/or one or more other processing units. Memory 159 can include a first primary storage, typically a random-access memory (RAM), and a second primary storage area, typically a non-volatile storage such as solid-state drive (SSD) or hard disk drive (HDD). Memory 159 stores programming instructions and data for processes operating on processor(s) 158. Primary storage typically includes basic operating instructions, program code, data and objects used by processor(s) 158 to perform their functions. Primary storage devices (e.g., memory 159) may include any suitable computer-readable storage media, described below, depending on whether, for example, data access needs to be bi-directional or uni-directional.

Sensor data database 152 includes data received from mobile sensor platforms 102A, 102B and/or 102C. After capture by mobile sensor platform 102A, 102B and/or 102C, sensor data stored in sensor data database 152 may be operated on by various analytics, as described below. Sensor data database 152 (e.g., position data database) stores position data received from mobile sensor platforms 102A, 102B and/or 102C. In some embodiments, sensor data database 152 stores position data as well as sensor data. In such embodiments, sensor data database 152 may be omitted. Server 150 may include other databases and/or store and utilize other data. For example, server 150 may include calibration data (not shown) used in calibrating sensors 110, 120 and 130.

System 100 may be used to capture, analyze, and provide information regarding hyper-local environmental data. Mobile sensor platforms 102A, 102B and 102C may be used to traverse routes and provide sensor and position data to server 150. Server 150 may process the sensor data and position data. Server 150 may also assign the sensor data to map features corresponding to the locations of mobile sensor platforms 102A, 102B and 102C within the same time interval as the sensor data was captured. As discussed above, these map features may be hyper-local (e.g., one hundred meter or less road segments or thirty meter or less road segments). Thus, mobile sensor platforms 102A, 102B and 102C may provide sensor data that can capture variations on this hyper-local distance scale. Server 150 may provide the environmental data, a score, confidence score and/or other assessment of the environmental data to a user. Thus, using system 100 hyper-local environmental data may be obtained using a relatively sparse network of mobile sensor platforms 102A, 102B and 102C, associated with hyper-local map features and processed for improved understanding of users.

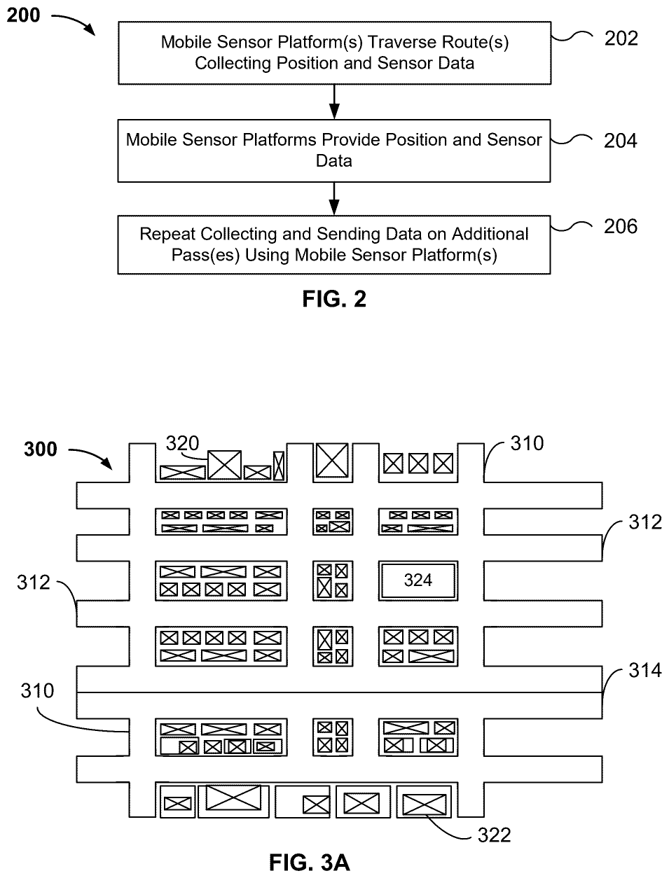

FIG. 2 depicts an exemplary embodiment of method 200 for capturing environmental data using mobile sensor platforms, such as mobile sensor platforms 102A, 102B and/or 102C. Method 200 is described in the context of system 100, but may be performed using other systems. For clarity, only some portions of method 200 are shown. Although shown in a sequence, in some embodiments, processes may occur in parallel and/or in a different order.

Mobile sensor platforms traverse routes in a geographic region, at 202. While traversing the routes, the mobile sensor platforms collect not only sensor data, but also position data. For example, a mobile sensor platform may sense one or more of NO2, CO, NO, O3, SO2, CO2, CH4, VOCs, particulate matter, other compounds, radiation, noise, light, and other environmental data at various times during traversal of the route. Other environmental characteristics, including but not limited to temperature, pressure, and/or humidity may also be sensed at 202. In addition, the time corresponding to the environmental data is also captured. The time may be in the form of a timestamp for the sensor data (sensor timestamp), which may correspond to a particular time interval. Different sensors on the mobile sensor platform may capture the environmental data at different times and/or at different frequencies. Also, at 202 the mobile sensor platforms capture position data, for example via a GPS unit. The position data may include location (as indicated by a GPS unit), velocity and/or other information related to the geographic location of the mobile sensor platform. In some embodiments, position data from other sources, such as acceleration, may be captured from by the vehicle or another source. The position data may include a timestamp (position timestamp) or other indicator of the time at which the position data is captured.

The mobile sensor platforms provide the position and sensor data to a server, at 204. In some embodiments, mobile sensor platforms provide this data substantially in real time, as the mobile sensor platforms traverse their routes at 202. Thus, the position and sensor data may be transmitted wirelessly to the server. In some embodiments, some or all of the position and/or sensor data is stored at the mobile sensor platform and provided to the server at a later time. For example, the data may be transferred to the server when the mobile sensor platform returns to its base. In some embodiments, the mobile sensor platform may process the sensor data and/or position data prior to sending the sensor and/or position data to the server. In other embodiments, the mobile sensor platform provides little or no processing. The sensor data and position data may be sent at the same time or may be sent separately.

At 206, the route traversal and data collecting of 202 and data sending of 204 are repeated. Thus, the mobile sensor platforms may traverse the same or different routes at 206. In either case, multiple passes of the same geographic locations, and thus multiple passes of the same corresponding map features, are made at 206. In some embodiments, the repetition at 206 may be periodic (e.g., approximately every week, month, or other time period). In some embodiments, the repetition at 206 may be performed based on other timing. In some cases, the same mobile sensor platform is sent on the same route and/or collects data for the same map features. In some embodiments, different mobile sensor platforms collect data may be used for the same routes and/or map features. Also at 206, steps 202 and 204 may be performed multiple times. Thus, at 206, data for a particular region may be aggregated over time.

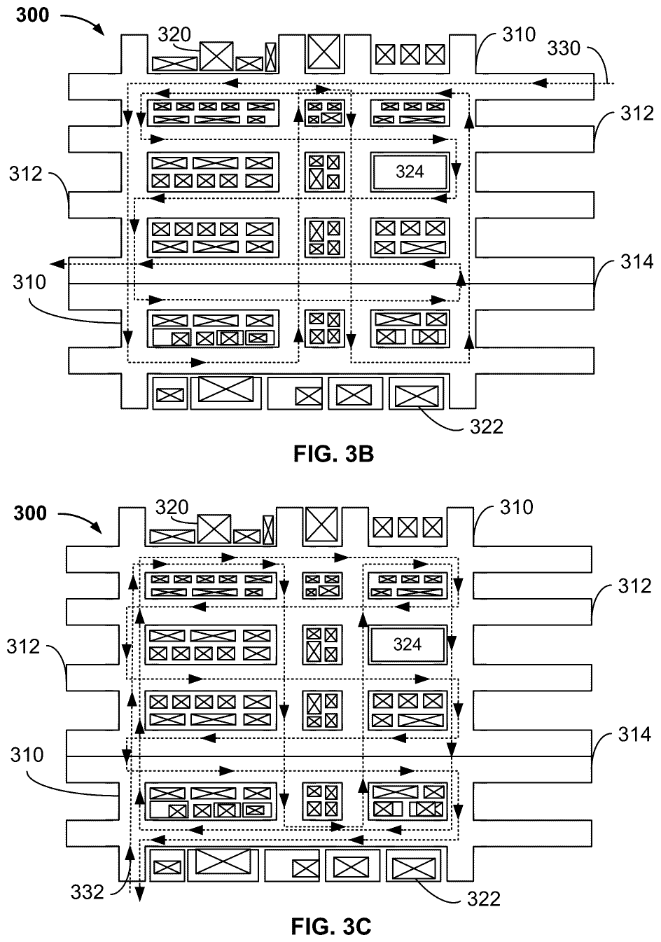

For example, FIGS. 3A-3C illustrate a particular geographic region and the routes that may be traversed using method 200. A map 300 corresponding to the geographic region is shown in FIG. 3A. Map 300 may be an open-source map or generated by another mapping tool. Map 300 includes streets 310 (oriented vertically on the page) and 312 (oriented horizontally on the page); larger street/highway 314, structures 320 and 322 and open area 324.

For simplicity, only one of each structure 320 and 322 is labeled. Open area 324 may correspond to a park, vacant lot, or analogous item. As can be seen in FIG. 3A, the density and size of structures 320 and 322 vary across map 300. Similarly, the density and size of streets 312, 314 and 320 also varies. In addition, structures 322 are more clearly separated by open regions, which may correspond to a yard or analogous area.

FIG. 3B illustrates map 300 as well as route 330 that may be traversed by a mobile sensor platform, such as mobile sensor platform 102A. At 202, mobile sensor platform 102A may traverse route 330. As can be seen in FIG. 3B, the route 330 includes a portion of each street 312 and 314 in map 300. Some portions of some streets are traversed multiple times for the same route 330. In some embodiments, this is still considered a single pass of these streets. As mobile sensor platform 102A traverses route 330 at 202, sensor data is captured by sensors 110, 120 and 130. Also at 202, position data is captured by position unit 145 throughout route 330. In some embodiments, the vehicle carrying mobile sensor platform 102A travels sufficiently slowly while traversing route 330 that sensor data and position data can be accurately captured for particular position(s). In some embodiments, mobile sensor platform 102A travels at a velocity that allows for multiple sensor data points for each map feature. Mobile sensor platform 102A also sends position and sensor data to server 150 at 204. This may be done while mobile sensor platform 102A traverses route 330 or at a later time. Other mobile sensor platforms 102B and/or 102C may also traverse the same or different routes and send data to server 150 at 202 and 204. Thus, multiple mobile sensor platforms may be used in method 200.

At 206, mobile sensor platform 102A and/or other mobile sensor platform(s) 102B and 102C repeat the route traversal, data collection and sending of the position and sensor data. In some cases, mobile sensor platform(s) 102A, 102B and/or 102C follow route 330 again. In some cases, mobile sensor platform(s) 102A, 102B and/or 102C traverses a different route. For example, FIG. 3C depicts map 300 with another route 332. As part of 206, mobile sensor platform(s) 102A, 102B and/or 102C may traverse route 332, collecting position and sensor data at 206 (repeating 202). In some embodiments, the vehicle carrying mobile sensor platform(s) 102A, 102B and/or 102C travels sufficiently slowly while traversing route 332 that sensor data and position data can be accurately captured for particular position(s). In some embodiments, mobile sensor platform(s) 102A, 102B and/or 102C travels at a velocity that allows for multiple sensor data points for each map feature (described below). Mobile sensor platform(s) 102A, 102B and/or 102C send sensor and position data to server 150 at 206 (repeating 204) during or after traversing route 330 and/or route 332.

Thus, using method 200, sensor and position data may be captured for regions of a map. The sensor data and position data may be provided to server 150 or other component for processing, aggregation, and analysis. Sensor data and position data are sensed sufficiently frequently using method 200 that variations environmental quality on the hyper-local scales may be reflected in the sensor data. Method 200 may be performed using a relatively small number of mobile sensor platforms. Consequently, efficiency of data gathering may be improved while maintaining sufficient sensitivity in both sensor and position data.

For example, FIGS. 3A-3C illustrate a particular geographic region and the routes that may be traversed using method 200. A map 300 corresponding to the geographic region is shown in FIG. 3A. Map 300 may be an open-source map or generated by another mapping tool. Map 300 includes streets 310 (oriented vertically on the page) and 312 (oriented horizontally on the page); larger street/highway 314, structures 320 and 322 and open area 324. For simplicity, only one of each structure 320 and 322 is labeled. Open area 324 may correspond to a park, vacant lot, or analogous item. As can be seen in FIG. 3A, the density and size of structures 320 and 322 vary across map 300. Similarly, the density and size of streets 312, 314 and 320 also varies. In addition, structures 322 are more clearly separated by open regions, which may correspond to a yard or analogous area.

FIG. 3B illustrates map 300 as well as route 330 that may be traversed by a mobile sensor platform, such as mobile sensor platform 102A. At 202, mobile sensor platform 102A may traverse route 330. As can be seen in FIG. 3B, the route 330 includes a portion of each street 312 and 314 in map 300. Some portions of some streets are traversed multiple times for the same route 330. In some embodiments, this is still considered a single pass of these streets. As mobile sensor platform 102A traverses route 330 at 202, sensor data is captured by sensors 110, 120 and 130. Also at 202, position data is captured by position unit 145 throughout route 330. In some embodiments, the vehicle carrying mobile sensor platform 102A travels sufficiently slowly while traversing route 330 that sensor data and position data can be accurately captured for particular position(s). In some embodiments, mobile sensor platform 102A travels at a velocity that allows for multiple sensor data points for each map feature. Mobile sensor platform 102A also sends position and sensor data to server 150 at 204. This may be done while mobile sensor platform 102A traverses route 330 or at a later time. Other mobile sensor platforms 102B and/or 102C may also traverse the same or different routes and send data to server 150 at 202 and 204. Thus, multiple mobile sensor platforms may be used in method 200.

At 206, mobile sensor platform 102A and/or other mobile sensor platform(s) 102B and 102C repeat the route traversal, data collection and sending of the position and sensor data. In some cases, mobile sensor platform(s) 102A, 102B and/or 102C follow route 330 again. In some cases, mobile sensor platform(s) 102A, 102B and/or 102C traverses a different route. For example, FIG. 3C depicts map 300 with another route 332. As part of 206, mobile sensor platform(s) 102A, 102B and/or 102C may traverse route 332, collecting position and sensor data at 206 (repeating 202). In some embodiments, the vehicle carrying mobile sensor platform(s) 102A, 102B and/or 102C travels sufficiently slowly while traversing route 332 that sensor data and position data can be accurately captured for particular position(s). In some embodiments, mobile sensor platform(s) 102A, 102B and/or 102C travels at a velocity that allows for multiple sensor data points for each map feature (described below). Mobile sensor platform(s) 102A, 102B and/or 102C send sensor and position data to server 150 at 206 (repeating 204) during or after traversing route 330 and/or route 332.

Thus, using method 200, sensor and position data may be captured for regions of a map. The sensor data and position data may be provided to server 150 or other component for processing, aggregation, and analysis. Sensor data and position data are sensed sufficiently frequently using method 200 that variations environmental quality on the hyper-local scales may be reflected in the sensor data. Method 200 may be performed using a relatively small number of mobile sensor platforms. Consequently, efficiency of data gathering may be improved while maintaining sufficient sensitivity in both sensor and position data.

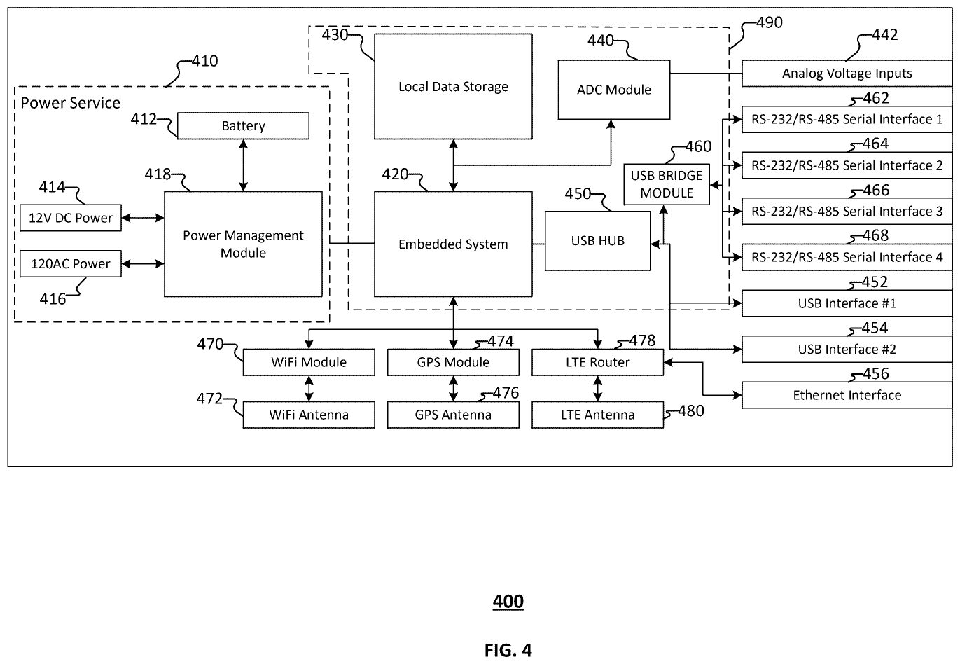

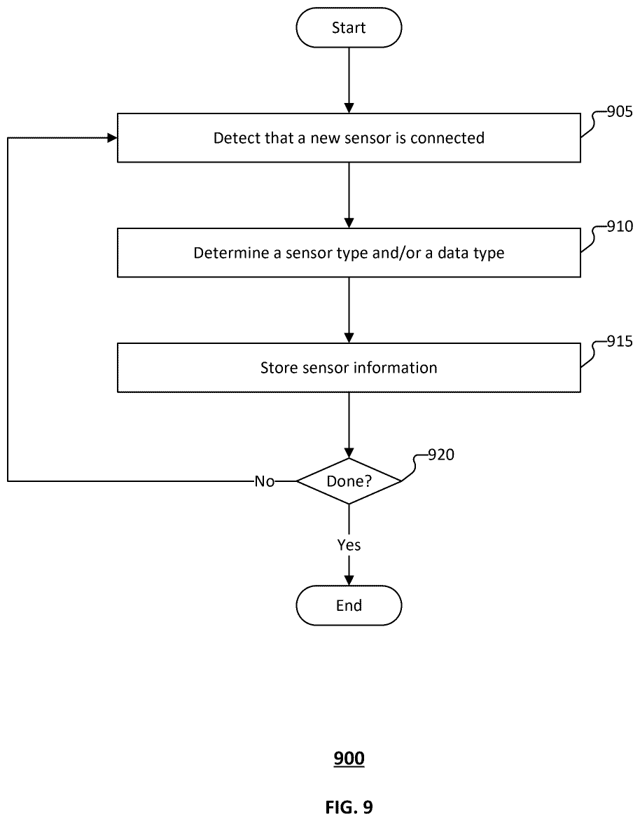

FIG. 4 illustrates an embodiment of an edge system for collecting and analyzing environmental data. In some embodiments, system 400 is implemented by, or integrated with, system 100 of FIG. 1. In some embodiments, system 400 implements one or more of processes 900-1100 of FIGS. 9-11.

The system operates as a robust and versatile middleware layer for environmental monitoring, designed to streamline the flow of data between diverse sensors and downstream analytics or visualization platforms. In some embodiments, the system captures (e.g., collects, receives, etc.) raw sensor outputs (e.g., sensor data), translates these readings into a predefined or universal format, and then stores them locally alongside contextual metadata such as timestamps, geolocation, and sensor identifiers. Because it performs the bulk of its data parsing and analysis at the edge, the system allows for rapid anomaly detection, timely operational responses, and a reduced dependence on external cloud services or networks. This local-first approach is especially beneficial for sites with limited connectivity or where real-time insights are critical/desirable.

The system is configured to automatically recognize and configure newly connected sensors. As an example, the system can configure the newly connected sensors without manual intervention. The system can determine which sensor is in use and what type of environmental data it provides based at least in part on data-level indicators (e.g., unique firmware identifiers) and/or physical characteristics (e.g., port types, port interface, or cable configurations, etc.). In some embodiments, if the sensor cannot be clearly identified through these automated methods, the system prompts the user or administrator to specify the missing information or to otherwise input a sensor definition for the newly connected sensor. Once the sensor type is established, the software applies a standardized parsing procedure, storing all incoming readings under a consistent structure. This automatic sensor detection and configuration technique greatly simplifies the adoption of new technologies or sensor models, as the system continuously adapts to new devices.

In addition to data ingestion and storage capabilities, the system is configured with local analytics capabilities that enable real-time evaluation of environmental conditions (e.g., to analyze the environmental data in real-time based on the sensor data). The system can be further configured to deploy machine learning models to spot patterns or forecast future values from ongoing measurements, enabling prompt/real-time intervention in scenarios like sudden spikes in pollutants or alerts about equipment failures. By eliminating the need to route data to offsite servers or wait on round-trip processing times, the system provides a vital advantage in situations that demand quick action and on-the-spot decision-making.

These comprehensive features, ranging from automated sensor detection to on-edge analytics, make the system a flexible and powerful solution for modern environmental monitoring applications. The system configuration ensures that diverse streams of sensor data are efficiently processed, intelligently assessed, and readily available for both real-time actions and long-term reporting or research.

In the example shown, system 400 comprises a power service 410 and data hub 490. System 400 further comprises various ancillary modules, such as communication modules, modules that obtain/provide contextual data, and communication interfaces via which environmental sensors can be coupled to system 400.

According to various embodiments, system 400 (or more specifically data hub 490) is deployed between the sensor layer and a server-based analytics or storage layer, thereby effectively serving as an intermediary that manages data flow and processing functions. Acting as a middleware, system 400 collects sensor outputs (e.g., obtains sensor data from connected environmental sensors), interprets or transforms the raw data into standardized formats (e.g., to obtain environmental data), and then makes the environmental data available for local analysis (e.g., real-time analysis and visualization). System 400 can further make the environmental data and/or raw sensor data available for further analysis or ingestion by higher-level services, such as cloud services or remote servers. Because system 400 resides at the edge, system 400 can process incoming sensor data in real time, reducing the necessity for sending every reading to a remote server or cloud platform. The ability for system 400 to provide local real-time analysis and/or visualization at the edge not only curtails bandwidth usage but also enables real-time responses to critical changes in the monitored environment.

Within an environmental data system, system 400 is configured to ensure that various sensing devices (e.g., environmental sensors) can all be brought online quickly, regardless of their individual data protocols or output characteristics. Upon detecting a new sensor connection, system 400 can determine a data profile, an identifier(s), and metadata from the sensor, the cable type/identifier used to connect the sensor to system 400, and/or the connection port. System 400 can then use an internal ruleset or configuration file(s) to decipher the sensor's data streams. Once the sensor's data type is known, system 400 can harmonize/associate the sensor data with contextual data, such as the time stamps, geolocation data, etc. In some embodiments, system 400 adjusts the sensor data (or the environmental data corresponding to the sensor data translated to a universal data format) for offset or calibration parameters. By performing these steps locally, system 400 streamlines downstream analytics, which may take place locally at system 400, in an environmental monitoring platform, or be visualized on a display (e.g., a display connected locally to system 400, or a display on a client system connected to system 400, such as via Wi-Fi module 470).

According to various embodiments, system 400 unifies the communication and data processing requirements at a local level, thereby enabling a standalone solution that can function even when network connectivity is unreliable or unavailable. This characteristics makes system 400 especially suited for remote environmental sites where real-time data analytics are crucial/desirable. Organizations can use system 400 to gather detailed time series data from multiple sensors, label and index the data according to uniform conventions, and store or forward the enriched data for advanced analytics locally at system 400 for more advanced analytics via a cloud platform or remote server. As a result, the overall environmental monitoring service becomes more resilient, flexible, and cost-effective, at least partly because the burden of manually configuring/defining sensors and/or intensive cloud or server-side processing is minimized.

Returning to the example shown, system 400 comprises power service 410. System 400 uses power service 410 to manage power delivery for various components, such as embedded system 420, local data storage 430, and communication modules. In some implementations, power service 410 supplies power for one or more environmental sensors connected to system 400. In the example shown, power service 410 comprises one or more power inputs, such as battery 412, DC power supply 414 (e.g., configured to provide 12V DC power), and/or AC power supply 416 (e.g., configured to provide 120 AC power). Power service 410 further comprises power management module 418, which manages the power for system 400.

Data hub 490 is configured to perform one or more of (a) detect a newly connected sensor(s), (b) determine a sensor type and/or environmental data type (e.g., the type of data the sensor collects and provides to system 400) for the newly connected sensor(s), (c) receive sensor data from one or more environmental sensors, (d) pre-process the sensor data (e.g., convert the sensor data to environmental data in a universal/pre-defined data format), (e) store the environmental data (e.g., the translated sensor data) and/or the raw sensor data locally to local data storage 430, (f) store context data in association with the environmental data and/or the raw sensor data, (g) perform real-time analysis (e.g., environmental analysis to obtain environmental analysis data or results) with respect to the environmental data and/or the associated context data, (h) identify/detect in real time environmental events/risks based at least in part on the environmental data and/or the associated context data, (i) generate a visualization based at least in part on the environmental data (e.g., to visualize the environmental analysis data or results from the real-time analysis), (j) provide the visualization, such as by causing a display (e.g., a display connected to system 400, or a display on a client system connected to system 400), (k) receive a user input with respect to the environmental data, such as a request to perform an active measure, (l) cause an active measure to be implemented (e.g., send an alert to a user or organization, request resources for remediation activities, etc.), and/or (m) communicate/upload the environmental data and/or context data or raw sensor data to a cloud platform or remote storage/database.

In the example shown, data hub 490 comprises embedded system 420, local data storage 430, analog-to-digital converter (ADC) module 440, USB hub 450 (e.g., an optically isolated USB hub), and/or USB bridge module 460. Embedded system 420 may comprise one or more processors configured to perform the functionality of the data hub 490, such as the sensor data collection, processing, and analysis. The one or more processors may comprise one or more of a central processing unit (CPU), a graphics processing unit (GPU), a data processing unit (DPU), an infrastructure processing unit (IPU), a function accelerator card (FAC), a network attached processing unit (NAPU), a field programmable gate array (FPGA), a vision processing unit (VPU), etc. Embedded system 420 further comprises one or more interfaces via which embedded system 420 connects to or otherwise communicates with other modules of data hub 490 and/or various communication interfaces (e.g., Wi-Fi module 470, GPS module 474, and/or long term evolution (LTE) router 478.

Local data storage 430 stores locally environmental data, context data, and/or raw sensor data. Local data storage 430 may additionally store environmental analysis data (e.g., results from performing analysis with respect to the environmental data), generated visualizations, etc. Local data storage 430 may comprise one or more memories to which the data is stored. For example, to reduce the latency in processing collected sensor data, the one or more memories include one or more high speed local memories, such as dynamic random access memory (DRAM), a double data rate memory (DDRx), embedded DRAM (eDRAM), SDRAM, and high bandwidth memory (HBM) of DRAM, etc. Various other types of memories may be implemented.

Data hub 490 comprises one or more modules via which sensor data is collected from sensors connected to system 400 via one or more interfaces. The collected sensor data can be provided to embedded system 420 for processing, analysis, and/or storage.

In some embodiments, data hub 490 comprises USB hub 450, which receives sensor data from sensors connected to system 400 via one or more USB interfaces such as USB interface 1 452, USB interface 2 454, etc.

In some embodiments, data hub 490 comprises ADC module 440 to collect raw voltage outputs from one or more connected sensors, such as voltage inputs received by analog voltage inputs interface 442. ADC module 440 enables system 400 to ingest raw signals in voltage form, and ADC module 440 can convert the raw voltage sensor data to meaningful data that can be further analyzed locally to provide real-time insights. By installing an ADC at the edge, system 400 can interface with a broader range of sensors that transmit analog signals, rather than being limited to devices that already convert their readings into a digital format. When a sensor is plugged into the designated ports, system 400 automatically samples the electrical signals at a specified rate (e.g., a rate that is mapped to a particular sensor type or particular environmental data type), capturing information such as voltage fluctuations that correspond to environmental or physical measurements. This conversion process produces a stream of numerical values that can be easily parsed by the system's real-time analysis routines.

By performing analog-to-digital conversion locally, system 400 ensures that environmental variables such as noise, signal degradation, or voltage drifts are accounted for in real time, rather than adding potential error during transportation to a cloud platform or a remote data center for processing. Because system 400 can control the sampling rate and resolution of ADC module 440, system 400 can tailor the conversion process to the specific type of measurement being collected, whether that involves high-frequency signals or lower-speed sensors. According to various embodiments, once the data is digitized, system 400 applies the same sensor detection, calibration, and analysis steps as it would for digital inputs, effectively unifying both analog and digital sensors in a single data-processing pipeline.

In some embodiments, data hub 490 further comprises a USB bridge module 460 that is configured to receive sensor data from one or more sensors connected via other interfaces (e.g., non-USB interfaces). The USB bridge module 460 may be connected to embedded system 420 via USB hub 450. In some embodiments, USB bridge module 460 enables system 400 to interface with sensors or instruments that communicate through RS-232, RS-485, or other serial standards. By connecting these devices to the USB bridge (e.g., via serial interface 1 462, serial interface 2 464, serial interface 3 466, and/or serial interface 4 468), the system (e.g., system 400, USB bridge module 460, etc.) effectively converts the raw serial signals into a USB data stream that system 400 (e.g., embedded system 420) can readily interpret. This technique allows system 400 to ingest data from a wider range of legacy or industrial sensors, many of which rely on older protocols yet still offer valuable measurements for environmental monitoring. The USB bridge module 460 thus acts as a physical gateway that simplifies the cabling requirements and ensures compatibility with more modern hardware platforms.

Once the serial signals are translated (e.g., via USB bridge module 460) into a USB data stream, the system (e.g., embedded system 420) parses the incoming packets according to the same universal or predefined format employed for other sensor inputs. System 400 (e.g., embedded system 420) reads any embedded identifiers, checks voltage patterns when applicable, and references configuration files to classify the type of sensor data. After identifying the sensor connected via USB bridge module 460, the system continues with its usual workflow of attaching/associating context metadata such as time stamps or geolocation, normalizing the readings, and storing them for further analysis. Whether the sensor outputs come from RS-232, RS-485, or another serial protocol, the USB bridge module 460 abstracts away the complexities of voltage levels and signal framing, allowing system 400 (e.g., embedded system 420) to treat the resulting data as though it originated from any other digital source.

By leveraging a USB bridge module 460, system 400 remains highly adaptable, supporting both modern and legacy devices without significant modifications to its core architecture. This system configuration is particularly advantageous in field environments or industrial settings where older sensors still generate critical data but have not been upgraded to support network-based or newer digital interfaces. The streamlined connection and automated data processing capabilities thus enable organizations to integrate a range of environmental sensors in a cost-effective manner, extending the overall usefulness and reach of the system.

System 400 comprises one or more modules (e.g., communication module) to receive sensor data and/or context data. In the example shown, system 400 comprises Wi-Fi module 470 that enables system 400 to communicate via Wi-Fi, such as to upload data to a cloud platform or remote database, or to connect to a client system to provide visualizations or receive user input. System 400 further comprises GPS module 474 that obtains geolocation data via GPS antenna 476. System 400 may comprise an LTE router 478 that is used to communicate with other systems (e.g., a cloud platform, remote database or server, a client system, etc.). LTE router 478 may be further configured to send/receive data from a system or device connected via Ethernet interface 456. For example, LTE router 478 may receive sensor data via a sensor connected via Ethernet interface 456.

According to various embodiments, system 400 (e.g., embedded system 420) automatically identifies and classifies connected sensors by examining a variety of characteristics associated with their signals and hardware connections. When a new sensor is attached, system 400 (e.g., embedded system 420) scans for any recognizable attributes, such as data patterns or communication protocols, to determine if the sensor's output matches a known profile (e.g., a data profile). This known profile might include information about how data values are delimited, the sampling rate, or specific byte sequences that correspond to a particular sensor's identity. By comparing the incoming data to this reference library of profiles, system 400 can infer the sensor type and the measurements the sensor is intended to collect.

Another mechanism according to which system 400 (e.g., data hub 490, embedded system 420, etc.) may discern sensor type (or environmental data type) is by monitoring the physical components/characteristics of the connection itself, such as the particular cable used or the interface via which the sensor is connected (e.g., the input slot into which the sensor is plugged). Different sensors often require distinct cable configurations or specialized terminals to handle voltage levels or analog-to-digital conversion requirements. By looking at these physical distinctions, system 400 (e.g., embedded system 420) narrows down which sensor (or sensor type) is likely attached. Additionally (and simultaneously), or alternatively, system 400 can read any metadata or identifier transmitted by the sensor as part of the data stream, providing an additional layer of verification or discernment. If the sensor's firmware includes a unique identifier, system 400 (e.g., embedded system 420) can align that identifier with entries in an internal lookup table to confirm both the sensor type and any calibration data or measurements (e.g., an environmental data type) it is designed to collect.

In some embodiments, system 400 (e.g., data hub 490, embedded system 420, etc.) uses voltage signatures in connection with identifying a sensor, sensor type, and/or environmental data type. Many sensors operate at designated voltage ranges or exhibit characteristic signals when sampling begins. System 400 can monitor these signals in conjunction with the initial data frames to confirm that the expected voltage thresholds match the profile of the sensor it has tentatively identified. If there is a mismatch, system 400 can either flag a possible error or continue scanning until it finds a matching sensor profile. Through this combination of physical and data-level checks, system 400 efficiently abstracts the sensor detection process from the end user. Rather than having to write custom code or manually configure each sensor, the user can simply plug in the device and allow system 400 to automatically complete the setup. Once the sensor is recognized, system 400 starts ingesting measurements under the correct configuration, ensuring seamless integration with downstream data analysis or visualization tools.

In some embodiments, system 400 (e.g., data hub 490, embedded system 420, etc.) implements a matching of incoming data stream to data profiles in connection with identifying a sensor, sensor type, and/or environmental data type. For example, system 400 matches the incoming data stream to a stored profile that represents how particular sensors transmit their measurements. These profiles may include details such as the length of the data packets, the frequency with which the sensor sends updates, and specific delimiters or metadata that appear in the transmitted data. By continuously monitoring the incoming data received from a sensor, system 400 can compare what it receives to a library of known data patterns. When system 400 finds a match, it classifies the sensor as a specific type (or the environmental data type) without relying on manual intervention.

In some embodiments, system 400 (e.g., data hub 490, embedded system 420, etc.) evaluates the hardware connection via which a sensor is connected in connection with identifying a sensor, sensor type, and/or environmental data type. For example, system 400 detects the type of sensor by evaluating the sensor data (e.g., metadata, data formatting, type of data, data profile, etc.), which can provide clear physical clues about the sensor's identity or sensor type. Some sensors have proprietary connectors or require specialized cables that correspond to a certain communication protocol. If the system detects that a sensor is plugged into a specific port designed for that protocol, it can infer the sensor's possible identity. This inference becomes stronger if system 400 determines the voltage levels or signal patterns on that cable line up with a known sensor's operating characteristics, further reinforcing that the sensor has been correctly identified.

In some embodiments, system 400 (e.g., data hub 490, embedded system 420, etc.) uses any digital information provided by the sensor itself in connection with identifying a sensor, sensor type, and/or environmental data type. Many modern sensors transmit an identifier as part of their data stream or include a firmware signature that announces the sensor brand, model, or other key details. System 400 reads this embedded identifier and cross-references it with an internal lookup table. If the identifier is recognized, system 400 immediately sets up the correct data parsing routine, ensuring that the measurements are interpreted accurately. If the identifier is not recognized, system 400 can fall back on additional/alternative detection strategies such as cross-validating the data pattern or voltage profile before labeling the sensor.

In some embodiments, system 400 (e.g., embedded system 420) is configured to perform sensor calibration at the edge rather than relying solely on post-processing steps that typically occur after data has been uploaded to a server or cloud. By integrating calibration into the local computing environment, system 400 can compare outputs from different instruments in real time and generate correction factors that align the results with known standards. When an instrument is connected to one port and a reference sensor or known calibration source is connected to another, the system can automatically analyze the two data streams, apply statistical methods to identify any discrepancies, and derive an appropriate calibration algorithm. This real-time comparison allows the user to quickly adjust sensor readings, ensuring that measurements from different devices can be interpreted with a common reference frame. In some embodiments, system 400 performs the calibration based at least in part on applying a predefined correction algorithm in real-time.

Users can also apply established calibration models directly from within system 400, a process that is especially helpful when dealing with widely used environmental metrics such as PM2.5. If a sensor measures particulate matter but outputs signals in raw voltages, system 400 can recognize that calibration data from a known source (e.g., such as PurpleAir or another standardized reference) should be applied to convert those raw voltages into an actionable concentration value. This approach eliminates the need for exporting data to another platform for manual calibration, allowing the operator to see fully corrected measurements in near real time on a local interface.

System 400 is configured to generate a user interface via which a user may interact with the system, such as to configure sensor settings/definitions or calibrations. For example, the user interface permits straightforward selection of calibration algorithms or correction tables, enabling operators to adapt the system to varied sensor models and operating conditions. In some embodiments, when a sensor is first plugged in, system 400 can prompt (e.g., embedded system 420 can configure a user interface to prompt) the user to identify any relevant calibration options or reference models, which are then stored in a local database for subsequent use. As more sensors are added or environmental conditions evolve, system 400 can continuously refine its calibration parameters to maintain accurate and consistent results. This dynamic calibration capability is particularly important when monitoring environmental conditions that are subject to frequent fluctuation or where multiple instruments are used for cross-checking results.

Incorporating ADC module 440 also enables system 400 to adapt calibration algorithms on the fly (e.g., in real-time) by comparing raw voltage readings to known reference voltages or calibration sources. When system 400 detects variability in the signal that falls outside expected ranges, it can adjust the calibration curves or coefficients to match the real-world performance of the sensor. This arrangement eliminates the need for specialized external hardware to handle voltage measurements, providing users with a more streamlined workflow. By seamlessly converting raw analog signals into actionable data, system 400 expands the scope of environmental monitoring to include a diverse set of sensors, all of which can be centrally managed and analyzed in near real time.

Because system 400 can enable calibration to occur in situ, operators can make real-time decisions based on the corrected measurements, improving the timeliness and reliability of environmental monitoring programs. Rather than waiting for hours or days for data to be uploaded, processed, and returned, system 400 quickly synchronizes raw sensor outputs with relevant calibration data to provide immediately meaningful information. This functionality delivers a robust and flexible edge solution that simplifies the overall workflow, reduces the likelihood of errors introduced through manual post-processing, and ensures that a wide range of sensors can be integrated into a coherent, calibrated monitoring framework.

According to various embodiments, system 400 implements a configuration file in connection with interpreting sensor data. For example, system 400 (e.g., embedded system 420 and/or local data storage 430) stores the configuration file, which can be updated from time-to-time, such as by a firmware update, etc. System 400 relies on a configuration file that is indicative of (e.g., dictates) how incoming sensor data should be parsed, interpreted, and recorded. System 400 can further use the configuration file in connection with identifying a sensor type, an environmental data type being collected/provided by the sensor, etc. When a sensor is connected, the middleware (e.g., system 400, embedded system 420, etc.) checks for any identifying information in the data stream or the physical connection and matches it to entries in the configuration file. Because this configuration file represents a comprehensive specification, it essentially teaches (e.g., specifies to) the system how to handle various types of sensor outputs, whether the data arrives in human-readable text format, binary packets, or raw voltage readings converted through ADC module 440. By consulting the configuration file, the middleware (e.g., system 400, embedded system 420, etc.) can determine the nature of the readings (e.g., whether the readings/sensor data correspond to temperature, particulate matter, or another environmental metric) and apply the correct algorithms for data extraction, calibration, and storage.

According to various embodiments, a benefit from the techniques implemented by system 400 is the configuration file employing a consistent format for describing sensor data, thus unifying the way different types of sensor outputs are ingested and processed. Whether the specification is encoded in JSON-LD or a Python script, the middleware (e.g., system 400, embedded system 420, etc.) relies on the same conceptual framework to read and interpret the data (e.g., the sensor data being ingested by system 400). During the ingestion process, system 400 can also attach (e.g., associate the sensor data with) contextual metadata, such as timestamps, geolocations, or sensor identification numbers, to the measurements. As a result, for example, each entry in the data pipeline carries both the raw sensor reading and relevant information about the circumstances under which it was collected, thereby aiding subsequent analyses.

According to various embodiments, over time, new sensor models or updated sensor firmware may become available, requiring changes to how the data is handled. In such cases, users can update the system's behavior by flashing a revised or expanded configuration file directly to the local device. This update technique avoids the need to rewrite large portions of the system's codebase, for example, because the middleware (e.g., system 400) only needs to consult the updated file to learn how to process the incoming signals. By making these updates available locally, system 400 remains operational even in settings with limited connectivity, thereby enabling field operators to adapt quickly to new sensor deployments or calibration needs without extensive downtime.

The use of a single, flexible specification file (e.g., the configuration file) enables the system to perform sensor identification, data interpretation, and contextual tagging that can be automated for virtually any environmental monitoring scenario. This technique not only simplifies the user experience but also offers robust adaptability in situations where sensors must be rapidly added, replaced, or reconfigured. Because the key logic is housed in a single reference source (e.g., the configuration file), maintenance and upgrades are greatly streamlined, and the sensor/environmental data analysis platform evolves with the environmental data collection requirements.

According to various embodiments, system 400 implements (e.g., locally deploys) one or more machine learning models in connection with identifying sensors (e.g., based on the ingested sensor data) or environmental data type, or to perform local analysis of the sensor data. System 400 is configured to accommodate machine learning models that run directly on the edge device (e.g., data hub 490), allowing sensor data to be parsed, evaluated, or analyzed without relying solely on external servers or cloud platforms. By integrating lightweight models into the local software environment, system 400 can classify incoming signals, detect anomalies, or perform rapid time-series forecasting in real time. This is particularly useful in environmental monitoring, where immediate (e.g., real-time) insights may be necessary for triggering alerts or informing on-site decisions.

Because the system already ingests and organizes sensor outputs, the sensor data (or environmental data corresponding to a converted sensor data, for example, for a particular environmental data type) is readily available for feeding into local machine learning workflows. For instance, if the user has uploaded a model trained to identify abnormal chemical concentration spikes, the system can continuously run newly ingested data (e.g., the collected sensor data or the environmental data corresponding to converted sensor data) through that machine learning model and flag anomalous readings as soon as they appear. Likewise, if the goal is to predict future sensor readings based on historical measurements, system 400 can apply lightweight forecasting models at the edge, giving operators timely projections without requiring a round-trip to a remote data center.

According to various embodiments, this edge-based approach (e.g., the local collection and analysis of sensor data) minimizes (e.g., reduces) latency and reduces dependency on network connectivity, making it suitable for remote or bandwidth-constrained deployments. In the context of machine learning model implementation for processing/analyzing sensor data, by keeping model inference on the local device (e.g., at the data hub 490), system 400 can also ensure greater data privacy and security, because sensitive information does not need to leave the operational environment. Additionally, any updates to these lightweight models deployed locally at system 400 can be pushed to the device (e.g., to data hub 490). This enables rapid adaptation to evolving monitoring needs or new algorithmic techniques, with minimal disruption to day-to-day data collection.

According to various embodiments, the techniques described herein allow for collecting and analyzing sensor data locally at the edge (e.g., broad-area monitoring) and combining analysis of this targeted collection with environmental data collected by a targeted monitoring, such as environmental data collected via a mobile sensor platform. In some embodiments, the system is designed to seamlessly integrate with both mobile environmental platforms and static monitoring installations, creating a unified view of large-scale environmental conditions. Through its ability to collect and interpret sensor outputs from a variety of sources, the edge device can aggregate real-time data from mobile platforms (e.g., such as vehicles or portable monitoring stations) while concurrently gathering measurements from stationary sensors positioned at strategic locations. The system can merge these inputs into a coherent data set, thereby enabling organizations to analyze broad area parameters and correlate them with more targeted, site-specific indicators. This functionality is especially useful in scenarios where a single vantage point is not sufficient, or where local anomalies require high-resolution tracking alongside regional trends.

For targeted area monitoring, the mobile sensor platforms may gather data on PM2.5, black carbon, nitrogen oxides (NO and NO2), ozone (O3), carbon monoxide (CO), carbon dioxide (CO2), methane, ethane, and total volatile organic compounds (VOCs). These measurements help build a comprehensive picture of general air quality across large geographic regions, detecting trends and fluctuations that are not necessarily visible from fixed-position sensors. Meanwhile, the system's integration with an organization's static monitoring network allows it to incorporate additional parameters specific to that location. Examples include specialized readings for heavy metals like arsenic, lead, and chromium, as well as PM fractions such as PM2.5, PM10, and ultrafine particles. Speciated VOCs, ammonium, chloride, ethylene oxide, or other chemicals relevant to local industrial activities may also be tracked, providing deeper insight into environmental stressors that require closer scrutiny.

According to various embodiments, as the system ingests diverse streams of data, it uses its edge-based computing capabilities to normalize the different formats, apply calibration factors, and cross-reference observations with contextual metadata such as timestamps and GPS coordinates. This multi-pronged approach allows for integrated analyses that extend beyond the capabilities of earlier systems that relied heavily on post-processing or single-sensor data sources. By focusing on real-time analytics, the platform can quickly detect hotspots of pollution, trace spikes in specific contaminants, and link those findings to broader trends identified by the mobile surveys. This synergy between mobile and static measurements ultimately makes it possible to deliver richer, more accurate insights into environmental conditions, equipping stakeholders with the information they need to respond effectively to emerging threats or compliance requirements.

Before this system, organizations typically struggled to combine mobile environmental data and targeted measurements from fixed sites into a single pipeline that supports real-time situational awareness. Either the data would be locked in separate databases, or it would require lengthy offline processes to align timestamps and formats. By contrast, this edge system automatically ingests both data types, converting raw sensor outputs into a predefined structure that enables immediate analysis. Through this harmonization, it becomes feasible to examine heavy metal concentrations in the same framework as broad-scale particulate matter or greenhouse gas levels, leading to a more holistic understanding of an area's environmental challenges and aiding in the planning of mitigation or research efforts.

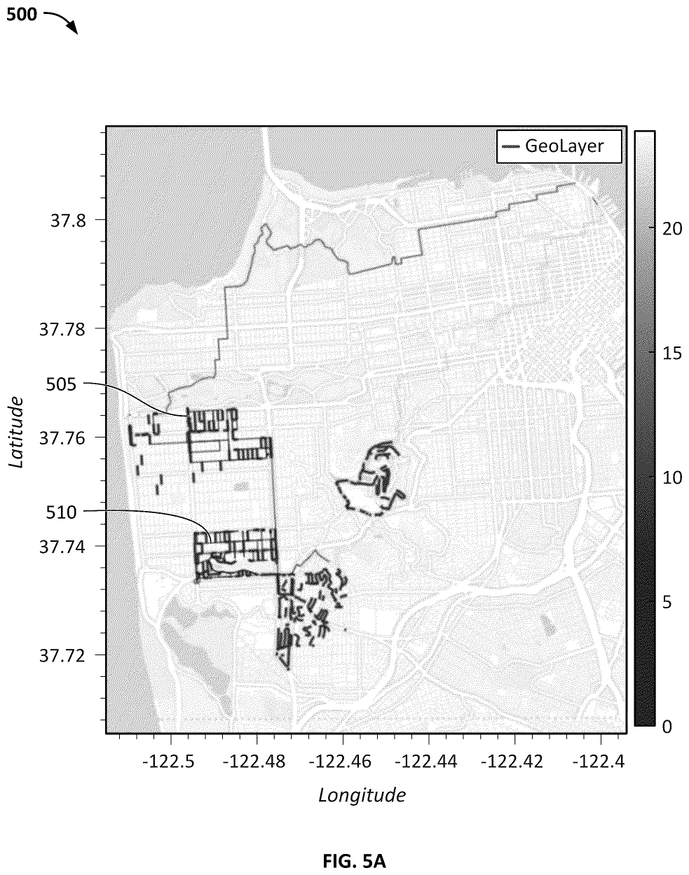

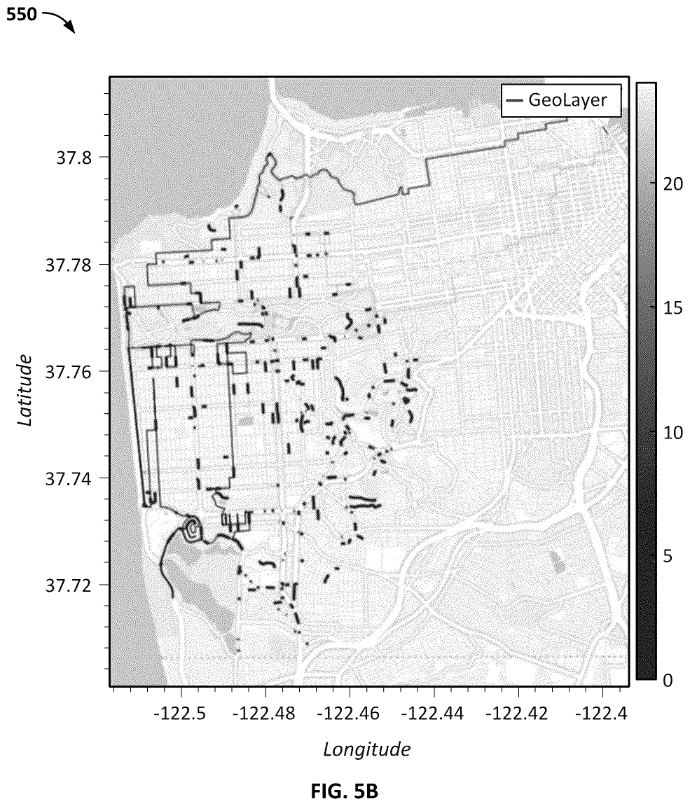

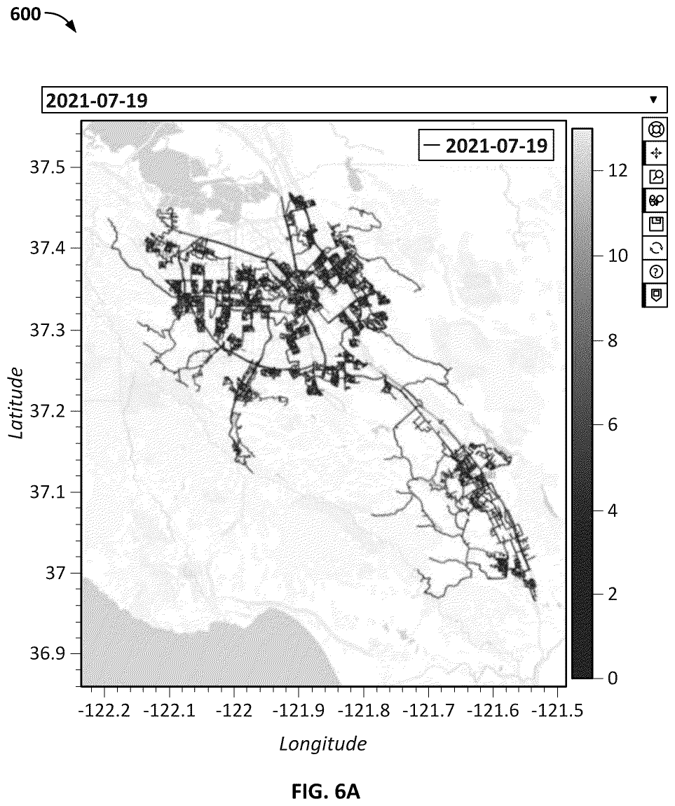

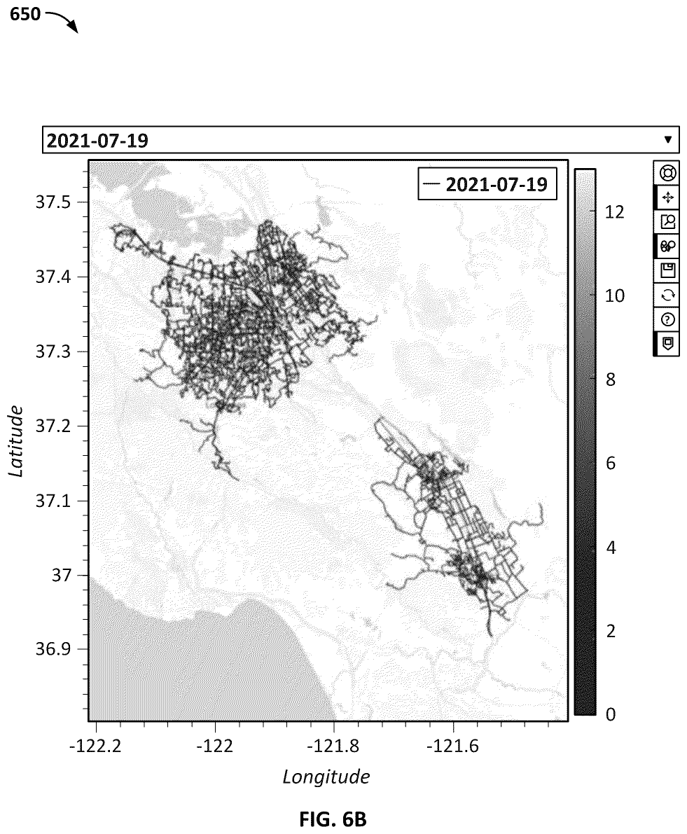

FIGS. 5A, 5B, 6A, and 6B illustrate examples of data collection paths. In the examples shown, FIGS. 5A and 6A show a map of driving routes or a sampling of a geographic region based on driving road segments within selected polygons/census tracts in the geographic region. In contrast, FIGS. 5B and 6B show a map of driving routes or a sampling of a geographic region based on driving a subset of randomly selected road segments within the geographic region.

In some embodiments, the system collects environmental data (e.g., sensor data collected from mobile sensors) via a mobile sensor platform. For example, the mobile sensor platform may be controlled to collect environmental data along the data collection paths. Various techniques for using a mobile sensor platform to sample data over a region may be implemented. Such techniques are further described in U.S. patent application Ser. No. 18/242,740, which is hereby incorporated herein in its entirety for all purposes.

In the example shown, in FIG. 5A, map 500 illustrates the sampling of environmental data during a session based on selection of polygons from a set of predefined polygons. As illustrated, polygons 505 and 510 are sampled during the session. The data collection was focused specifically on polygons 505 and 510, thereby providing little data diversity across the entire geographic region. The emphatically displayed lines within the polygons are indicative of the roads along which the environmental data was sampled. As shown, the data collection according to a selection of polygons from a set of predefined polygons results in the collection along each road within the selected polygons but a dearth of data collection in the remaining areas of the geographic region.

In the example shown in FIG. 6A, map 600 illustrates a sampling of environmental data using the same selection process as the sampling illustrated in map 500. Map 600 provides a representation of the sampling/coverage over a simulated 19 days of driving (e.g., 19 different sessions or a set of sessions for a plurality of vehicles). Map 600 shows that sampling within the region is very focused on a small set of polygons/areas that each have a very dense environmental data collection. However, map 600 shows many areas within the geographic region with no environmental data collection thereby resulting in a poor representation of environmental data over the entire geographic region.

Collecting environmental data based on the assignment of OSM ways includes assigning each OpenStreetMap (OSM) way a pass count based on the count of passes for which environmental data has been collected (e.g., over a predefined period of time, such as a month, year, or contract length, etc.) on the road segment(s) for the OSM ways. In some embodiments, the system randomly selects a road segment(s) for which environmental data is to be collected. As an example, the selection of the road segment(s) is selected randomly with a probabilistic weight that is proportional to the pass count for the corresponding road segment/OSM way. The weighting of the road segments is assigned so that the OSM way having a lowest corresponding pass count (e.g., a lowest number of data points in the historical environmental data) is the most likely to be selected.

Collection of environmental data by selecting polygons (e.g., census tracts) and directing vehicles to drive all or most of the road segments within the selected polygons during a corresponding session can result in spatial artifacts caused because a subset of areas of the geographic region corresponding to the polygons in which evaluation data is collected may be oversampled, and a subset of areas of the geographic region may be under sampled. To improve the spatial diversity of evaluation data sampling in the geographic region, the system may select and assign certain road segments for a session to ensure that a smaller percentage of road segments are driven in any one polygon/census tract, but a larger percentage of polygons/census tracts are sampled (e.g., through driving a subset of road segments within more polygons). However, the selection, grouping, and ordering of the road segments for collection of evaluation data during a set of sessions can still be inefficient. Although the process improves the spatial variance in sampled locations across the geographic region (e.g., reduces/eliminates the spatial artifacts within the map of the geographic region populated with environmental data), both processes used to sample the geographic regions corresponding to maps 500 and 600, and the geographic regions corresponding to maps 550 and 650 may result in temporal artifacts. For example, both processes may oversample certain road segments at certain times of day and under sample other road segments at certain times of day.

In the example shown, in FIG. 5B, map 550 illustrates the sampling of environmental data during a session based on selection of specific road segments. As illustrated, the sampling along the set of road segments performed during the session provides a good spatial diversity of air quality measurements. In the example shown in FIG. 6B, map 650 provides a representation of a simulated sampling over the geographic region during 19 driving days. Map 650 illustrates a very high spatial diversity among air quality measurements relative to map 600.

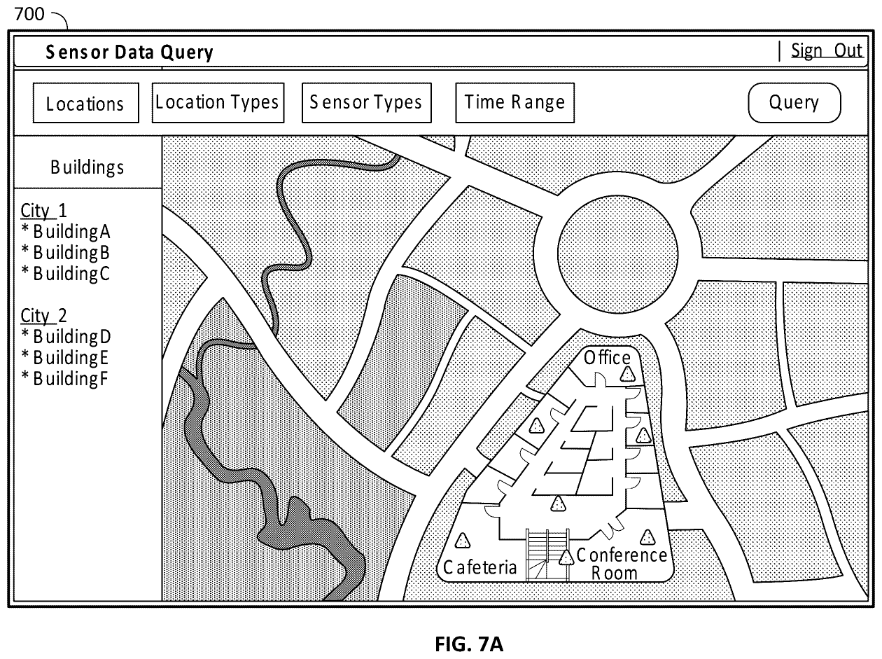

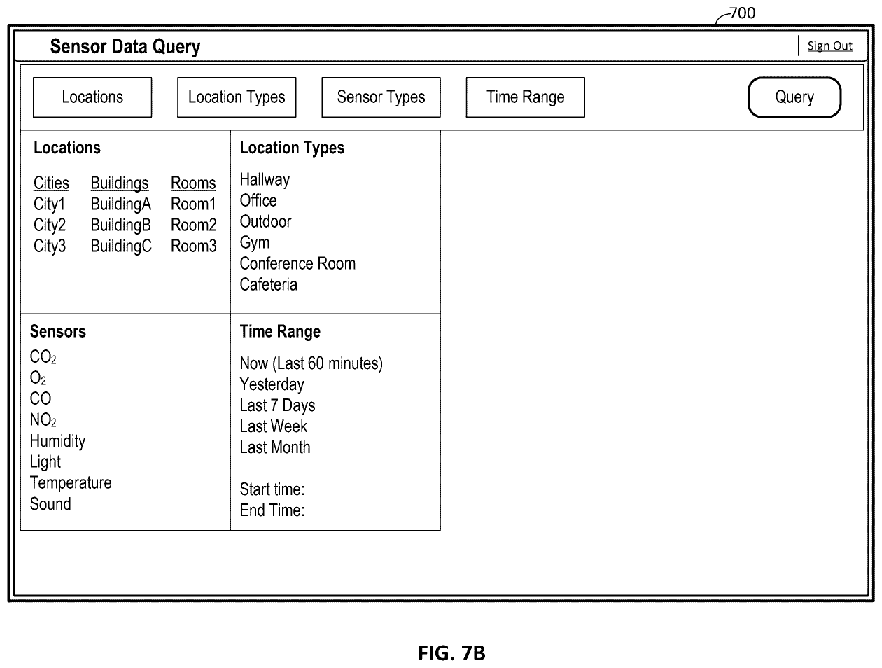

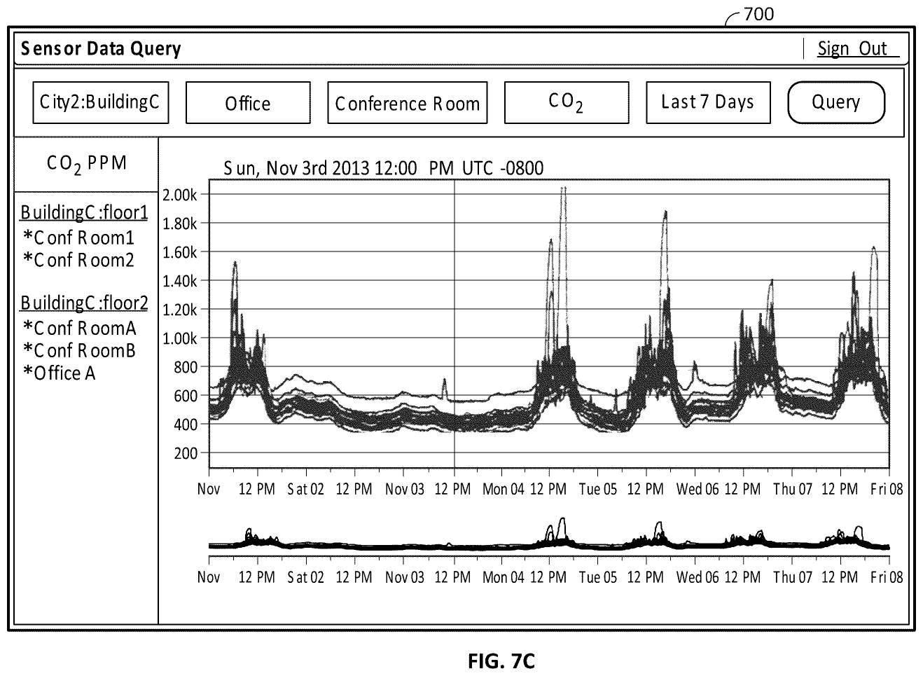

FIGS. 7A-7C illustrate examples of user interfaces for displaying visualizations of environmental data based on real-time analysis of sensor data collected at an environmental data hub according to various embodiments. According to various embodiments, the system configures a user interface via which a user can view environmental data and environmental analysis data based on the local analysis of sensor data collected at an environmental data hub. The user interface may be displayed on a display connected to the environmental data hub or to a client system (e.g., a tablet, a mobile phone, a laptop, etc.) connected to the environmental data hub.

In the present example, the user interface 700 provides selection parameters including locations, location types, sensor types, and time range. A user can input selections or parameters for an analysis to be performed or for environmental data to be visualized. In the present illustration, “locations” refer to geographic locations or a place such as cities, buildings within each city, parks, or other structures within each city, and “location types” refer to different types or kinds of premises, such as hallway, office, outdoor, gym, conference room, and cafeteria. In the present illustration, “sensors” refer to air quality sensors such as CO₂, O₂, CO, NO₂, and also environmental sensors such as humidity, light, temperature, and sound sensors. However, according to various embodiments, various other types of environmental sensors may be implemented. In the present illustration, “time range” includes a set of pre-determined time intervals of interest, such as Now (the most recent 60 minutes), Yesterday, Last 7 Days, etc. Custom date and time range can also be entered.

Using the user interface 700 to formulate a query request, the system can receive via the user interface a location selection from a list of locations. In one embodiment, the “location” parameter has a default value of all locations selected. Thus, when no location selection is made, the mechanism for visualizing environmental data selects all the locations. Locations can be specified by a city name and further defined by a building name within each city. In some embodiments, the system configures the user interface to present a map image or a graphical display of geographic locations in the user interface to aid in the location selection.

In some embodiments, the environmental data hub is deployed at a particular building or campus. Location types in this context can include categories of location types, such as hallway, office, outdoor, gym, conference room, and cafeteria. In some embodiments, the system configures the user interface to present a map image or a graphical display in the user interface to aid in the selection of the location types or a specific location type. For example, the map image may display the floor plan of a building with the locations of the deployed sensor shown by a sensor icon/representation. A selection of one or more location types can be made by clicking on the sensor icons.

In one embodiment, the “sensor type” parameter has a default value of all sensors selected. Thus, when no sensor type selection is made, the mechanism for visualizing environmental data selects all the sensors available. For example, the list of sensors can include carbon dioxide (CO₂) sensors, oxygen (O₂) sensors, carbon monoxide (CO) sensors, temperature sensors, and humidity sensors. Various other types of environmental sensors may be implemented, such as local environmental sensors connected to the environmental data hub, or environmental sensors deployed on a mobile sensor platform that provides a more targeted monitoring.

The user interface 700 can provide a list of pre-determined time ranges that are of common interest, such as Now (the most recent 60 minutes), Yesterday, Last 7 Days, etc. A custom date and time range can also be entered by specifying the start time and the end time. In one embodiment, the “time range” parameter has a default value of “Last 7 days” selected. Thus, when no time range selection is made, the mechanism for visualizing environmental data selects sensor data for the last 7 days.

An example of a query result is illustrated in FIG. 7C. In response to a user selection for a query to visualize analyzed environmental data, the system can locally analyze collected environmental data (e.g., via sensor data from a plurality of locally connected environmental sensors). In the example, a query request is made for the CO₂ sensor data for City2, BuildingC. The selected location types include Office and Conference Room. The sensor data query method interprets the request as a logical OR operation and displays sensor data for all offices and all conference rooms in BuildingC of City2. The selected time frame is Last 7 Days and thus the sensor data query method presents the sensor data for CO₂ for the last 7 days in the offices and conference rooms of BuildingC of City2. The sensor data display has a fixed vertical scale (2.00K ppm for CO₂ sensor). As can be observed from the sensor data display, the conference rooms and offices are not occupied over the weekend and the rooms have low CO₂ levels during those days. But during the work week (Monday to Friday), the CO₂ level in the rooms becomes high in the afternoon hours of each day.

In some embodiments, the sensor data query method provides a meaningful display of sensor data to enable hypothesis driven inquiry. A search request can be formulated based on a hypothesis and the sensor data query method can be used to display and compare sensor data to evaluate the hypothesis.

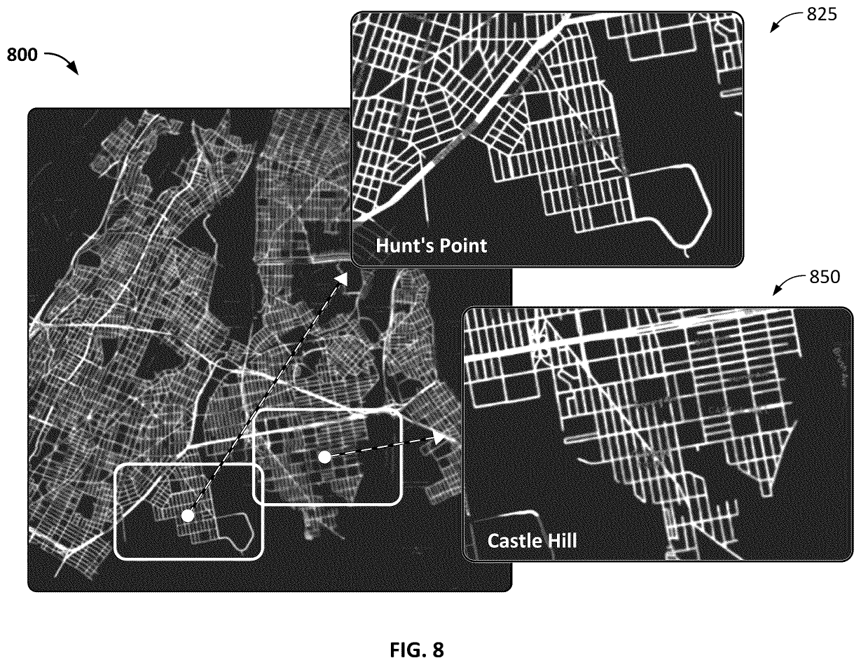

FIG. 8 illustrates an example of a visualization of data collected via a broad area monitoring of a predefined region according to various embodiments. In the example shown, environmental sensor data is used to collect broad area environmental data. The broad area environmental data can be collected by one or more environmental sensors, such as stationary sensors, deployed by an organization (e.g., a government regulatory agency, etc.) and connected locally to the environmental data hub described herein. User interface 800 visualizes this environmental data collected via a broad-area monitoring. In the example shown, the broad-area environmental data includes environmental data 825 and 850 corresponding to different regions (e.g., neighborhoods) over the broad-area being monitored. The system can query this broad-area environmental data for specific measurements to a particular location or region within the broad-area.

FIG. 9 illustrates a method for collecting environmental data at an edge system according to various embodiments. According to various embodiments, process 900 is implemented at least in part by system 400 of FIG. 4. In some embodiments, process 900 is implemented locally at an edge system that collects raw sensor data from a set of environmental sensors coupled thereto.

According to various embodiments, the system has the ability to detect newly connected sensors (e.g., environmental sensors) by continuously or periodically polling its communication interfaces, checking whether any hardware changes have occurred, or by analyzing the data streams that flow into the system. Whenever a signal (e.g., sensor data) is received that does not match the patterns of existing sensor outputs, the system investigates further to ascertain whether it originates from a device (e.g., an environmental sensor) that has not yet been registered. This technique may include established connection ports, comparing expected voltage or data rates, or reading embedded flags or metadata. Once the system identifies that a new sensor is likely connected, it initiates a discovery process to classify the type of data (e.g., the environmental data type) that sensor produces.

In some embodiments, to accomplish this classification, the system references a configuration file that outlines known sensor types and the corresponding environmental data each sensor provides. The file can include entries mapping specific ports or interfaces to particular sensor categories, as well as rules for interpreting identifiers within the data stream itself. The configuration file may also note which cable type or physical connector is typically used by each sensor model, enabling the system to cross-reference the hardware setup with the signal characteristics. By synthesizing all this information, the system arrives at an informed guess about what sort of sensor has been attached. Once identified, the sensor's data output is parsed and channeled into the standard environmental data pipeline, ensuring that the readings become consistent with other measurements.

In some cases, the system may fail to automatically classify a sensor based on its internal rules. As an example, this can happen when a new sensor does not match existing profiles in the configuration file, or if the sensor's data output contains unusual or insufficient metadata. In some embodiments, under these circumstances, the system prompts a user, administrator, or operator to supply any missing information. This might involve choosing from a list of known sensor types, manually specifying a new sensor definition, or confirming details about the measured environmental parameter. By providing this fallback mechanism, the system ensures that unrecognized devices can still be brought online and their data processed, while also improving its future ability to recognize and interpret similar sensors without operator intervention.

Returning to the example shown, at 905, the system detects that a new sensor is connected. At 910, the system determines a sensor type and/or a data type. At 915, the system stores sensor information. At 920, the system determines whether process 900 is complete. In some embodiments, process 900 is determined to be complete in response to a determination that no further environmental data is to be collected, an administrator indicates that process 900 is to be paused or stopped, etc. In response to a determination that process 900 is complete, process 900 ends. In response to a determination that process 900 is not complete, process 900 returns to 905.

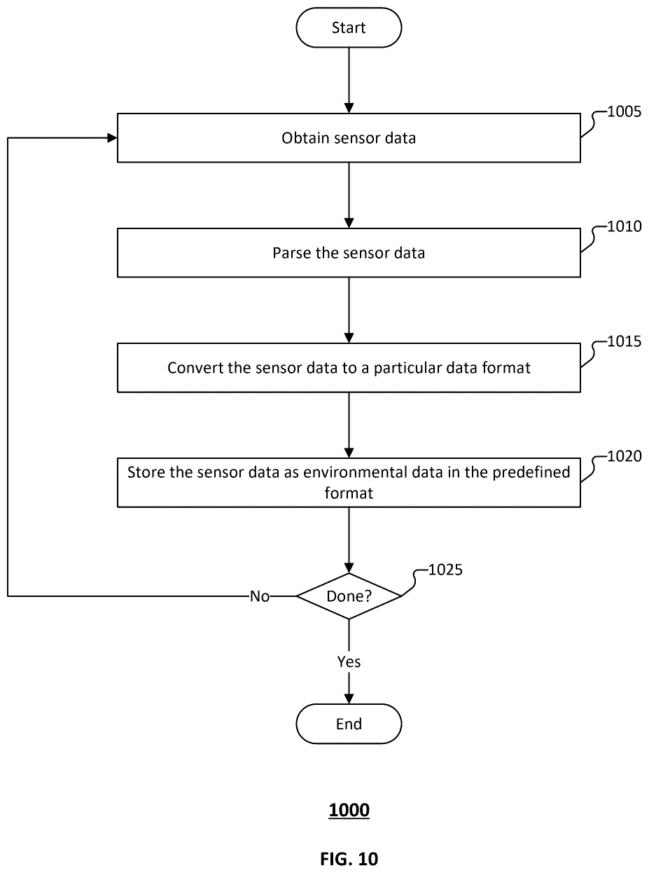

FIG. 10 illustrates a method for collecting and storing environmental data at an edge system according to various embodiments. According to various embodiments, process 1000 is implemented at least in part by system 400 of FIG. 4. In some embodiments, process 1000 is implemented locally at an edge system that collects raw sensor data from a set of environmental sensors coupled thereto.

According to various embodiments, the system begins by collecting raw data from various environmental sensors, capturing signals (e.g., sensor data) that could range from simple voltage outputs to complex digital data streams. These raw readings may comprise essential information about environmental measurements, such as temperature, humidity, chemical concentrations, or other metrics relevant to the monitoring context. Because different environmental sensors often produce outputs in inconsistent formats, in some embodiments, the system initially treats these signals (e.g., the sensor data) as unstructured or semi-structured inputs, gathering them into a buffer or temporary storage location until they can be further interpreted. During this data ingestion phase, the system continuously monitors the connections to detect when a new sensor comes online, allowing it to adapt dynamically as sensors are swapped or added.

Once the raw sensor data is aggregated, the system parses it according to a set of predefined rules and schemas. In some embodiments, the system parses the sensor data in real-time as it is collected from one or more environmental sensors. The parsing instructions comprised in the predefined rules and/or schemas can be stored in a configuration or firmware file that outlines how each sensor's data stream (or each sensor type or each environmental data type) should be dissected, whether that involves splitting incoming packets on specific delimiters or interpreting certain byte sequences as sensor identifiers. After the sensor data is parsed, the system converts the parsed sensor data into a universal format that encapsulates the measurement values and any relevant metadata, such as the units of measurement or the name of the monitored parameter. This transformation ensures that subsequent analyses (e.g., environmental analysis performed locally) or storage tasks handle each sensor reading with a consistent structure, no matter the original data source. In some embodiments, the system also stores the raw sensor data, which can be used to validate the locally performed environmental data analysis or to run further data analysis (e.g., by a cloud service or remote server).

In some embodiments, in parallel with parsing and format conversion, the system automatically attaches (e.g., associates) context data to each reading. That context data might include a precise timestamp from the system's own clock, geospatial coordinates obtained through GPS, an identifier for the sensor or site where the measurement originated, an identifier of a sensor type or environmental data type, etc. By embedding this contextual information at an early stage, the system creates an enriched dataset that can be readily queried, visualized, or analyzed. The universal, context-rich data is then stored locally, providing real-time access for tasks such as machine learning model inference, real-time visualization, or targeted queries that filter readings based on time or location.

Through ongoing observation of sensor outputs, the system can recognize when a new device (e.g., a newly connected environmental sensor) has been introduced by detecting patterns that do not match those of existing sensors or by reading embedded identifiers. In such cases, the system determine the new sensor type and the relevant environmental data type. For example, the system can determine the new sensor type and/or relevant environmental data type based at least in part on one or more of (a) a predefined configuration file, (b) a data profile, (c) an identifier comprised in the collected sensor data, (d) a port or interface via which the environmental sensor is connected to the system, and (e) a cable via which the environmental sensor is connected to the system. Once the system has classified the sensor, the system applies the appropriate parsing and conversion routines, seamlessly integrating the newly introduced device (e.g., the newly connected environmental sensor) into the established data pipeline. By automatically performing these tasks, the system streamlines the process of adding or replacing sensors, ultimately making environmental data collection more adaptable and efficient.

Returning to the example shown, at 1005, the system obtains sensor data. At 1010, the system parses the sensor data. At 1015, the system converts the sensor data to a particular data format. At 1020, the system stores the sensor data as environmental data in the predefined format. At 1025, the system determines whether process 1000 is complete. In some embodiments, process 1000 is determined to be complete in response to a determination that no further environmental data is to be collected, no further sensor data is to be processed, an administrator indicates that process 1000 is to be paused or stopped, etc. In response to a determination that process 1000 is complete, process 1000 ends. In response to a determination that process 1000 is not complete, process 1000 returns to 1005.

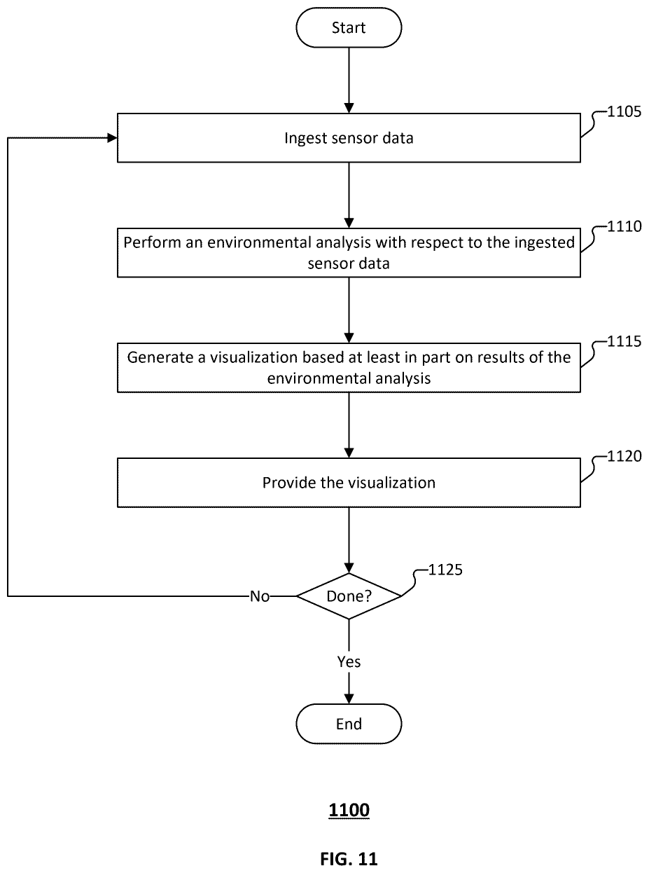

FIG. 11 illustrates a method for performing an environmental data analysis locally at an edge system according to various embodiments. According to various embodiments, process 1100 is implemented at least in part by system 400 of FIG. 4. In some embodiments, process 1100 is implemented locally at an edge system that collects raw sensor data from a set of environmental sensors coupled thereto.

The system ingests sensor data from one or more environmental monitoring devices (e.g., environmental sensors), collecting readings that may include temperature, chemical concentrations, particulate matter levels, or any other metric relevant to the application. In some embodiments, when these signals are collected, the system checks them against a predefined or universal format, ensuring that each new measurement is standardized and appropriately labeled. By converting raw sensor outputs into a consistent schema, the system facilitates efficient data integration and enables subsequent environmental analysis that treats each sensor reading equally, regardless of its original format or protocol. This standardized process is particularly critical in environments where multiple sensor types operate simultaneously, each with its own data structure.

Once the data is normalized, the system performs an environmental analysis that can encompass a range of tasks such as outlier detection, trend identification, or applying machine learning models to predict future measurements. This environmental analysis occurs locally (e.g., using local processors at the environmental data hub) and optionally in real time. The local environmental analysis can leverage edge-based algorithms to reduce latency and limit dependency on external computing resources. If the system detects any anomalies in the data, such as sudden spikes in pollutant levels, the system can flag them immediately (e.g., in real time), thereby allowing operators to take/request timely actions. In some embodiments, the system then organizes the results of this environmental analysis into a coherent data set, complete with timestamps, sensor identifiers, and relevant contextual information that can be referenced later for further study or compliance documentation.

According to various embodiments, the system is configured to generate (e.g., locally generate) visualizations that communicate the results of the environmental analysis. Whether the user is onsite at the environmental data hub or connected through a local network using Wi-Fi or Bluetooth, the system produces graphs, maps, or interactive dashboards that display results of the environmental analysis (e.g., environmental analysis data), which may include current and historical readings. This visualization can be accessed on a variety of client devices, such as tablets, mobile phones, or laptops, offering flexibility in how operators view and interact with the data. Because the system hosts the visualization tools locally, it can serve real-time graphs or geospatial overlays directly to a user (e.g., via a display connected to the environmental data hub or via a user's device), bypassing the need for constant Internet or remote service connectivity. By providing real-time visual feedback on sensor measurements and identified trends, the system empowers users to make informed decisions about environmental conditions without waiting for remote processing or post-collection analysis.

In some embodiments, the system generates a user interface that is designed to provide more than just passive data display. Alongside visualizations of sensor measurements and analysis results, the interface includes interactive control elements that enable the user to initiate various actions. For instance, when the environmental analysis data provided on the user interface shows a spike in pollutant levels on a graph or map, the user can click a button or toggle a switch to trigger an alert. This alert can be routed to relevant stakeholders, such as onsite personnel or remote monitoring teams, ensuring that potential environmental issues are brought to attention without delay.

Additionally, or alternatively, the user interface is configured to allow operators to request remediation activities directly from the visualization view, streamlining the process of responding to anomalies. As an illustrative example, if a sudden increase in water contaminants is observed, the user can select a control element that automatically sends a request to a maintenance or response crew. This request may include environmental analysis data (or raw sensor data) and/or context data, for example, the request may include location details, sensor readings, and timestamps, giving the remediation team all necessary information at a glance. In cases where the user wants to confirm the result of a suspicious reading, a dedicated control element on the user interface can be selected to re-run the analysis, updating the graphs or maps in near real time. By placing these interactive features within the same user interface as the environmental data, the system eliminates extra steps or the need to switch applications, allowing decisions and actions to occur more efficiently.

In some embodiments, the system configures the user interface to comprise control elements via which the user can manage administrative tasks, such as calibrating a sensor on-demand or adjusting the sampling rate to gather more frequent measurements. These controls are usually embedded near relevant visual components, so a user observing a potential drift in the data can promptly (e.g., immediately) recalibrate the device with just a few inputs to the user interface. Because all these capabilities are integrated into a single user interface, the system simplifies the operational workflow, providing a powerful and user-friendly environment for both real-time environmental monitoring and rapid response to emergent events.

Returning to the example shown, at 1105, the system ingests sensor data. In some embodiments, the system collects raw sensor data from one or more environmental sensors, normalizes the data (e.g., converts the sensor data to a predefined/universal data format), and stores the corresponding environmental data locally. At 1110, the system performs an environmental analysis with respect to the ingested sensor data. The system can perform real-time and local environmental analysis, such as according to a predefined routine or in response to a user input. At 1115, the system generates a visualization based at least in part on results of the environmental analysis. At 1120, the system provides the visualization. In some embodiments, the system provides the visualization to a user via a user interface. The user interface may be displayed on a display comprised at the edge system, and/or a display comprised in a client system (e.g., a tablet, a laptop, a mobile phone, etc.) that is connected to the edge system. In the case that the visualization is displayed at the client system, the system sends the visualization to the client system. At 1125, the system determines whether process 1100 is complete. In some embodiments, process 1100 is determined to be complete in response to a determination that no further environmental data is to be collected, no further sensor data is to be processed, an administrator indicates that process 1100 is to be paused or stopped, etc. In response to a determination that process 1100 is complete, process 1100 ends. In response to a determination that process 1100 is not complete, process 1100 returns to 1105.

Various examples of embodiments described herein are described in connection with flow diagrams. Although the examples may include certain steps performed in a particular order, according to various embodiments, various steps may be performed in various orders and/or various steps may be combined into a single step or in parallel.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A system, comprising:
a bridge configured to receive time series sensor data sets from a plurality of environmental sensors and having a plurality of formats; and
a data hub, coupled with the bridge, configured to parse each of the time series sensor data sets, determine a sensor type and data format for each of the plurality of environmental sensors, and convert corresponding sensor data to a predefined format.

2. The system of claim 1, wherein the bridge is further configured to be coupled to the plurality of environmental sensors.

3. The system of claim 1, wherein the time series sensor data sets comprise continuous sensor data sets.

4. The system of claim 1, wherein the predefined format is a universal format to which sensor data collected from a plurality of different environmental sensor types is converted.

5. The system of claim 1, wherein the data hub is further configured to store the corresponding sensor data as environmental data in the predefined format.

6. The system of claim 5, wherein the environmental data is stored in association with context data for the time series sensor data sets.

7. The system of claim 6, wherein the data hub is further configured to upload one or more of the environmental data and the context data to a cloud service or a remote database.

8. The system of claim 6, wherein the context data is stored in a JavaScript Object Notation for Linked Data (JSON-LD) data format.

9. The system of claim 6, wherein the context data comprises GPS data.

10. The system of claim 5, wherein the data hub is further configured to locally analyze the environmental data to obtain environmental analysis data.

11. The system of claim 10, wherein the data hub is further configured to locally generate a visualization based at least in part on the environmental analysis data.

12. The system of claim 10, wherein the data hub analyzes the environmental data in real-time.

13. The system of claim 10, wherein the data hub detects environmental events in real time based at least in part on local real-time analysis of the environmental data.

14. The system of claim 10, wherein the data hub converts the corresponding sensor data to the predefined format in real-time and compares in real time environmental data from a plurality of data streams from two or more of the plurality of environmental sensors.

15. The system of claim 1, wherein the data hub is configured to automatically recognize a new environmental sensor connected to the bridge, and to determine whether sensor data collected from the new environmental sensor is convertible to the predefined format.

16. The system of claim 1, wherein the time series sensor data sets comprise raw voltage data collected from one or more of the plurality of environmental sensors.

17. The system of claim 1, wherein the data hub is further configured to perform a calibration of at least one of the plurality of environmental sensors.

18. A method for sensing air quality with a sensor platform, comprising:
receiving, at an edge system, sensor data from a particular environmental sensor of a plurality of environmental sensors connected to the edge system;
determining an environmental data type based at least in part on one or more of the sensor data and sensor information detected based on a connection between the particular environmental sensor and the edge system;
parsing the sensor data based at least in part on the environmental data type; and
causing environmental analysis data to be displayed.

19. The method of claim 18, wherein the determining the environmental data type comprises determining an environmental sensor type and a sensor data format.

20. A computer program product for collecting and analyzing environmental data with an edge sensor platform, the computer program product being embodied in a tangible computer readable storage medium and comprising computer instructions for:
receiving, at an edge system, sensor data from a particular environmental sensor of a plurality of environmental sensors connected to the edge system;
determining an environmental data type based at least in part on one or more of the sensor data and sensor information detected based on a connection between the particular environmental sensor and the edge system;
parsing the sensor data based at least in part on the environmental data type; and
causing environmental analysis data to be displayed.
